(19) United States
(12) Patent Application Publication
(10) Pub. No.: US 2024/0164780 A1
(43) Pub. Date: May 23, 2024

(54) TISSUE REPAIR SCAFFOLDS WITH IMPROVED FEATURES FOR IMPLANTATION

(71) Applicant: Auxilium Biotechnologies Inc., San Diego, CA (US)

(72) Inventors: Aaron KEHRER, Ypsilanti, MI (US); Nathaniel JOHNSON, Ypsilanti, MI (US)

(21) Appl. No.: 18/241,746

(22) Filed: Sep. 1, 2023

Related U.S. Application Data
(63) Continuation of application No. PCT/US2022/021005, filed on Mar. 18, 2022.
(60) Provisional application No. 63/163,644, filed on Mar. 19, 2021.

(51) Int. Cl. A61B 17/11; A61F 2/00; A61L 27/18; A61L 27/52; A61L 27/58

(52) U.S. Cl. CPC A61B 17/1128; A61F 2/0077; A61L 27/18; A61L 27/52; A61L 27/58; A61F 2002/0081; A61F 2250/0067; A61F 2250/0097; A61L 2430/32

(57) ABSTRACT

Described herein are tissue repair scaffolds having improved features which aid in the installation of the scaffolds. Such features include alignment markers, exterior markers which denote the terminal points of interior channels, and increased sheath thickness.

### CROSS-REFERENCE TO RELATED APPLICATIONS

This application is a continuation of International Application No. PCT/US2022/021005 filed on Mar. 18, 2022 which claims the benefit of U.S. Provisional Application No. 63/163,644 filed on Mar. 19, 2021, both of which are incorporated herein by reference in their entirety.

### BACKGROUND

Nerve injuries are generally permanent injuries, and result in the loss of motor function and physical sensation. They are among the most devastating and debilitating injuries which can be suffered by an individual. Unfortunately, nerve injuries are a common result of trauma to nerves, and results in paralysis or disability of tens of thousands of individuals each year. There exists a need in the art for improvements to medical technology which can facilitate the healing of injuries to neural tissue as to restore motor function and physical sensation, and prevent the paralysis or disability of individuals suffering a nerve injury.

### SUMMARY OF THE INVENTION

One such device useful for the regeneration of neural tissue includes tissue repair scaffolds having microchannels designed for implantation into a subject. Such devices are useful for the treatment of physical damage and lesions of nerves. However, there exists a need for improved tissue repair scaffolds, including for nerve injury to spinal and peripheral nerves.

The nervous system has a capacity for regeneration after injury with the correct medical intervention. However, functional regeneration after injury is largely incomplete if the injured axons become misaligned or lose contact with innervated tissues, which commonly occurs in most cases of neural injury. Major functional deficits result and include deficient re-innervation of target tissues and painful neuroma formation. Current surgical treatments for repair of nerves involve placement of autologous nerve grafts. These grafts have many disadvantages, including donor site morbidity, limited supply of donor grafts, and time and complexity of surgery.

Experimental development of scaffolds to support peripheral nerve repair have resulted in commercially available nerve guides. However, many of these scaffolds have only a single, large diameter channel that results in misalignment of regenerating axons with their proper targets. Upon implantation in a transected rat sciatic nerve model, regenerating axons lose linear orientation along a proximal end shortly after entering the scaffold. This results in misorientation of the axons and pain due to neuroma. Further, there also exists a potential for failure of the implant due to error in surgical integration due to puncturing of the microchannel, or misalignment of the device and microchannel(s) therein. There exists a need in the art for improved neural tissue repair scaffolds to facilitate the regeneration of neural tissue.

Provided herein are tissue repair scaffolds (e.g., scaffolds for nerve repair, such as spinal or peripheral nerves) which have improved properties and features for implantation into a subject. The tissue repair scaffolds provided herein have one or more channels disposed within an exterior sheath. The microchannels are configured to allow a severed or damaged nerve to regrow through the device and recreate a severed or damaged neural connection. In some cases, the devices comprise a plurality of channels (“multichannel” devices), which provide the advantage of providing enhanced guidance for individual axons through each channel of the scaffold for an improved connection of a distal and proximal nerve end upon nerve reconnection.

In some aspects, the tissue repair scaffolds provided herein enable a surgeon to more readily, easily, and accurately insert the scaffold into a subject in need. In some embodiments, the tissue repair scaffolds have external markers that allow an implanting surgeon to readily ascertain if the scaffold is properly aligned (e.g., that one or more microchannels are in a linear configuration and that the scaffold has not become twisted). In some embodiments, the tissue repair scaffolds have an external marker which denotes where an overhang region begins, which allows the surgeon to suture a damaged or severed nerve end to the overhang without piercing the delicate interior microchannels and compromising the efficacy of the scaffold. In some embodiments, the tissue repair scaffolds have an increased sheath thickness compared to other multichannel nerve repair scaffolds (e.g., 500 micron), imbibing the scaffolds with increased strength and stability which prevents tearing of the scaffold during implantation and improved attachment to the nerve segment after implantation while the tissue regenerates.

In one aspect, provided herein, is a tissue repair scaffold comprising: a sheath having a first end and a second end; a microchannel disposed within an interior of the sheath, wherein the microchannel traverses the sheath from the first end to the second end; and a marker disposed on an exterior surface of the sheath running substantially from the first end to the second end.

In some embodiments, the marker comprises a groove. In some embodiments, the exterior surface of the sheath comprises at least 2, 3, or 4 grooves disposed thereon. In some embodiments, the exterior surface of the sheath comprises 4 grooves disposed thereon. In some embodiments, the grooves are about evenly dispersed about a circumference of the sheath. In some embodiments, the groove is substantially parallel with the microchannel. In some embodiments, the groove has a width or depth of at least about 50 microns.

In some embodiments, the tissue repair scaffold comprises a plurality of microchannels disposed within the interior of the sheath. In some embodiments, the plurality of microchannels are substantially parallel. In some embodiments, the marker is substantially parallel to each of the plurality of microchannels.

In some embodiments, the length of the scaffold is about 0.5 cm to about 30 cm. In some embodiments, the inner diameter of the sheath is about 1.5 mm to about 10 mm.

In some embodiments, the sheath, the microchannel, or both are made from a biodegradable polymer. In some embodiments, the biodegradable polymer is selected from polyethylene glycol, polyethylene glycol diacrylate, gelatin, methacrylated gelatin, collagen, methacrylated collagen, polylactic acid, poly glycolic acid, poly(lactic-co-glycolic acid), polycaprolactone, and acrylated polycaprolactone, or any combination thereof.

In some embodiments, walls of the microchannel comprise a plurality of pores disposed within the walls. In some embodiments, walls of the microchannel comprise hydrogel materials. In some embodiments, the sheath comprises an overhang structure which extends beyond the first end, the second end, or both.

In some embodiments, wherein the microchannel is configured to allow growth of nerve tissue. In some embodiments, the nerve tissue comprises a spinal nerve or a peripheral nerve.

In one aspect, provided herein, is a tissue repair scaffold comprising: a sheath having a first end and a second end, wherein the sheath has a thickness of at least about 250 microns; a plurality of microchannels disposed within an interior of the sheath, wherein the microchannels traverse the sheath from the first end to the second end.

In some embodiments, the sheath has a thickness from about 250 microns to about 700 microns. In some embodiments, the sheath has a thickness of about 300, about 350, about 400, about 450, about 500, about 550, or about 600 microns. In some embodiments, the sheath is made from a biodegradable polymer. In some embodiments, the biodegradable polymer is selected from polyethylene glycol, polyethylene glycol diacrylate, gelatin, methacrylated gelatin, collagen, methacrylated collagen, polylactic acid, poly glycolic acid, poly(lactic-co-glycolic acid), polycaprolactone, and acrylated polycaprolactone, or any combination thereof. In some embodiments, the biodegradable polymer is polycaprolactone. In some embodiments, the sheath has a porosity of about 40% vol to about 75% vol.

In some embodiments, the tissue repair scaffold comprises from about 7 to about 200 microchannels. In some embodiments, each microchannel has a wall thickness of about 10 microns to about 60 microns. In some embodiments, walls of the microchannels comprise a plurality of pores disposed within the walls. In some embodiments, walls of the microchannels comprise hydrogel materials. In some embodiments, the microchannels are hexagonal, round, triangular, rectangular, square, pentagonal, heptagonal, octagonal, nonagonal, decagonal, elliptical, or trapezoidal in shape. In some embodiments, the length of the scaffold is about 0.5 cm to about 30 cm. In some embodiments, the inner diameter of the sheath is about 1.5 mm to about 10 mm.

In some embodiments, the sheath comprises an overhang structure which extends beyond the first end, the second end, or both. In some embodiments, the microchannel is configured to allow growth of nerve tissue. In some embodiments, the nerve tissue is a spinal nerve or a peripheral nerve.

In one aspect, provided herein, is a tissue repair scaffold comprising: a microchannel having a first end and a second end; a sheath comprising: the microchannel disposed within an interior of the sheath; an overhang structure, wherein the overhang structure extends beyond the first end or the second of the microchannel; and a marker on an exterior of the sheath at a location that corresponds with the first end or the second end of the microchannel.

In some embodiments, the marker comprises a groove on the exterior of the sheath at the location that corresponds with the first end or the second end. In some embodiments, the groove circumferentially encompasses the exterior of the sheath. In some embodiments, the groove has a depth or width of at least about 50 microns. In some embodiments, the marker has a length of at least 200 microns.

In some embodiments, the tissue repair scaffold comprises an overhang structure over both the first end and the second end. In some embodiments, the overhang structure extends at least about 0.5 mm beyond the first end or the second end. In some embodiments, the overhang structure is configured to receive at least one suture.

In some embodiments, the sheath is made from a biodegradable polymer. In some embodiments, the biodegradable polymer is selected from polyethylene glycol, polyethylene glycol diacrylate, gelatin, methacrylated gelatin, collagen, methacrylated collagen, polylactic acid, poly glycolic acid, poly(lactic-co-glycolic acid), polycaprolactone, and acrylated polycaprolactone, or any combination thereof.

In some embodiments, the tissue repair scaffold comprises a plurality of microchannels. In some embodiments, the tissue repair scaffold comprises from about 7 to about 200 microchannels. In some embodiments, each microchannel has a wall thickness of about 10 microns to about 60 microns. In some embodiments, the microchannels are hexagonal, round, triangular, rectangular, square, pentagonal, heptagonal, octagonal, nonagonal, decagonal, elliptical, or trapezoidal in shape. In some embodiments, walls of the microchannels comprise a plurality of pores disposed within the wall. In some embodiments, walls of the microchannels comprise hydrogel materials. In some embodiments, the length of the scaffold is about 0.5 cm to about 30 cm. In some embodiments, the inner diameter of the sheath is about 1.5 mm to about 10 mm.

In some embodiments, the microchannel is configured to allow growth of nerve tissue. In some embodiments, the nerve tissue is a spinal nerve or a peripheral nerve.

In one aspect, provided herein, is a method of restoring nerve function comprising implanting a tissue repair scaffold provided herein into a nerve injury site in a subject in need thereof. In some embodiments, implanting the tissue repair scaffold allows restoration of nerve function across the injury site.

In some embodiments, implanting the tissue repair scaffold comprises suturing a first nerve end to the device such that the first nerve end regrows through the microchannel starting at the first end. In some embodiments, implanting the tissue repair scaffold further comprises suturing a second nerve end to the device such that the second nerve end regrows through the microchannel starting at the second end.

In some embodiments, the nerve is a peripheral or spinal nerve. In some embodiments, the nerve is fully or partially lesioned. In some embodiments, the nerve injury site comprises a gap between nerve ends from about 0.5 mm to about 10 cm.

Aspects disclosed herein provide a tissue repair scaffold comprising: a sheath having a first end and a second end; a microchannel disposed within an interior of the sheath, wherein the microchannel traverses the sheath from the first end to the second end; and at least one marker disposed on an exterior surface of the sheath.

In some embodiments, wherein the marker comprises a feature disposed on an exterior surface of the sheath running substantially from the first end to the second end. In some embodiments, wherein the marker runs along a length of the sheath and is parallel to the microchannel. In some embodiments, wherein the marker comprises a groove. In some embodiments, wherein the exterior surface of the sheath comprises at least 2, 3, or 4 grooves disposed thereon. In some embodiments, further comprising a plurality of markers, wherein the markers indicate the alignment of the device and/or torsion placed upon the device. In some embodiments, wherein the exterior surface of the sheath comprises 4 grooves disposed thereon. In some embodiments, wherein the grooves are about evenly dispersed about a circumference of the sheath. In some embodiments, wherein the groove is substantially parallel with the microchannel. In some embodiments, wherein the groove has a width or depth of at least about 50 microns. In some embodiments, wherein the tissue repair scaffold comprises a plurality of microchannels disposed within the interior of the sheath. In some embodiments, wherein the plurality of microchannels are substantially parallel. In some embodiments, wherein the marker is substantially parallel to each of the plurality of microchannels. In some embodiments, wherein the marker comprises an overhang structure, wherein the overhang structure extends beyond the first end or the second of the microchannel; and a marker on an exterior of the sheath at a location that corresponds with the first end or the second end of the microchannel. In some embodiments, wherein the marker comprises a groove on the exterior of the sheath at the location that corresponds with the first end or the second end. In some embodiments, wherein the groove circumferentially encompasses the exterior of the sheath. In some embodiments, wherein the groove has a depth or width of at least about 50 microns. In some embodiments, wherein the marker has a length of at least 200 microns. In some embodiments, wherein the tissue repair scaffold comprises an overhang structure over both the first end and the second end. In some embodiments, wherein the overhang structure extends at least about 0.5 mm beyond the first end or the second end. In some embodiments, wherein the overhang structure is configured to receive at least one suture. In some embodiments, further comprising a first plurality of markers and a second plurality of markers, wherein the first plurality of markers comprises two or more feature disposed on an exterior surface of the sheath running substantially from the first end to the second end, wherein the first plurality of markers runs along a length of the sheath and is parallel to the microchannel, wherein the second plurality of markers comprises an overhang structure, wherein the overhang structure extends beyond the first end or the second of the microchannel; wherein the second plurality of markers comprises a feature on an exterior of the sheath at a location that corresponds with the first end or the second end of the microchannel. In some embodiments, wherein the first plurality of markers and a second plurality of markers are configured to indicate the alignment of the microchannel therein, and the position of the microchannel relative to the adjacent nerve tissue.

Aspects disclosed herein provide a method of restoring nerve function comprising implanting the tissue repair scaffold described herein into a nerve injury site in a subject in need thereof.

In some embodiments, wherein implanting the tissue repair scaffold allows restoration of nerve function across the injury site. In some embodiments, wherein implanting the tissue repair scaffold comprises suturing a first nerve end to the device such that the first nerve end regrows through the microchannel starting at the first end. In some embodiments, wherein implanting the tissue repair scaffold further comprises suturing a second nerve end to the device such that the second nerve end regrows through the microchannel starting at the second end. In some embodiments, wherein the nerve is a peripheral or spinal nerve. In some embodiments, wherein the nerve is fully or partially lesioned. In some embodiments, wherein the nerve injury site comprises a gap between nerve ends from about 0.5 mm to about 10 cm. In some embodiments, further comprising aligning the marker at the first end at a proximal end of the injury site, and aligning the marker at the second end at a distal end of the injury site. In some embodiments, wherein the marker is aligned with a physiological correct direction of an axon to be regenerated across the injury site. In some embodiments, wherein implanting the tissue repair scaffold comprises inserting the scaffold into the injury site, utilizing the first plurality of markers to verify the alignment of the microchannel and to verify that the device is not placed under a torsion. In some embodiments, implanting the tissue repair scaffold comprises inserting the scaffold into the injury site, utilizing the second plurality of markers to verify the location of the microchannels relative to an adjacent nerve tissue and the position of the overhand relative to the adjacent nerve tissue. In some embodiments, wherein implanting the tissue repair scaffold comprises suturing the scaffold to an adjacent tissue without puncturing the microchannel. In some embodiments, wherein implanting the tissue repair scaffold comprises suturing the scaffold to an adjacent tissue in a parallel relative to alignment of a host axon, wherein the device is not placed under a torsion. In some embodiments, wherein regeneration of neural tissue is promoted as a result of one or more of: a) not puncturing the microchannel; or b) facilitating parallel alignment of the scaffold and alignment of an axon therein.

In other aspects, the present disclosure provides methods of making a biomimetic scaffold for promoting neural tissue growth by 3D printing. Scaffolds provided herein can be made using a variety of 3D printing techniques. Examples of 3D printing techniques which can be used to prepare 3D printed scaffolds include extrusion printing, inkjet printing, laser based-stereolithography, digital light processing stereolithography, and volumetric 3D printing (a.k.a. holographic 3D printing). In some embodiments, a biomimetic scaffold provided herein is prepared by digital light processing 3D printing. Additional methods of 3D printing of biomimetic scaffolds are described in PCT/US2017/065857, which is hereby incorporated by reference in its entirety.

In other aspects, the biomimetic scaffolds provided herein can be made from other techniques, including casting, molding, electrospinning, embossing, or any other suitable method. Exemplary alternative methods for preparing biomimetic scaffolds are described in, for example, PCT/US2020/027773, PCT/US2016/056104 and PCT/US2020/012966, each of which is incorporated by reference in its entirety.

### BRIEF DESCRIPTION OF THE FIGURES

The novel features of the methods and compositions described herein are set forth with particularity in the appended claims. A better understanding of the features and advantages of the present methods and compositions described herein will be obtained by reference to the following detailed description that sets forth illustrative embodiments, in which the principles of the methods and compositions described herein are utilized, and the accompanying drawings of which:

FIG. 1 shows a drawing of cross-sectional view of an exemplary tissue repair scaffold as provided herein.

FIG. 2 shows a drawing of an exterior side-view of an exemplary tissue repair scaffold as provided herein.

FIG. 3 shows a drawing of an exterior front-view (a view through the microchannels) of an exemplary tissue repair scaffold as provided herein.

FIG. 4 shows a drawing of an exterior view from an angled viewing position of an exemplary tissue repair scaffold as provided herein.

FIG. 5 shows a picture of an exterior side-view of an exemplary tissue repair scaffold as provided herein.

FIG. 6 shows a picture of an exterior front-view (a view through the microchannels) of an exemplary tissue repair scaffold as provided herein being held between the fingers of an individual.

### DETAILED DESCRIPTION

The nervous system has a great capacity for regeneration after injury. However, functional regeneration after injury is largely incomplete if the injured axons become misaligned or lose contact with innervated tissues. Major functional deficits result and include deficient re-innervation of target tissues and painful neuroma formation. Current surgical treatments for repair of nerves involve placement of autologous nerve grafts. These grafts have many disadvantages, including donor site morbidity, limited supply of donor grafts, and time and complexity of surgery.

Experimental development of scaffolds to support peripheral nerve repair have resulted in commercially available nerve guides. However, many of these scaffolds have only a single, large diameter channel that results in misalignment of regenerating axons with their proper targets. Upon implantation in a transected rat sciatic nerve model, regenerating axons lose linear orientation along a proximal end shortly after entering the scaffold. This results in misorientation of the axons and pain due to neuroma.

More recently, researchers have attempted to create nerve channels with multiple smaller “microchannels,” which allow individual axons to travel each in their own linear channels in order to better guide resulting connections. While this approach shows promise and provides advantages for enhancing the extent, rate, guidance, targeting and lesion-distance over which neural tissue (e.g., axons) can regenerate, the scaffolds create several challenges and complications in their installation.

For example, in some cases, multichannel scaffolds require that each of the interior channels be aligned and substantially parallel such that upon installation, the correct axonal connections are made. If the device becomes twisted or deformed during installation (a relatively simple occurrence owing to the small size and flexible nature of the device), the ability of the device to form the desired nerve connections can be compromised.

Additionally, in some embodiments, channels disposed within an interior of the device cannot be seen from an exterior vantage point of the device. Thus, during installation, it can be difficult for an implanting surgeon to ascertain where on the interior of the device the channels lie. This can be problematic because, in some cases, the tissue repair scaffold is sutured to a detached nerve through the use of an overhang structure on the device. Because the sheath of the devices is opaque (due to the materials used in their construction), there is a risk that the installing surgeon may suture the device to the relevant tissue at an improper part of the device and puncture the interior channels, thereby compromising the integrity of the channels and risking misalignment of the axons or other tissue.

Finally, in some instances, tissue repair scaffolds are sutured directly to relevant tissues (e.g., nerve tissue) through a sheath of the scaffold. In some cases, previous iterations of tissue repair scaffolds suffer from a lack of structural integrity either during or after implantation. In some instances, tissue repair scaffolds are made from biodegradable polymers that allow the scaffold to desirably break down after a period of time suitable for the desired tissue to regrow. However, in some instances, the exteriors of such scaffolds, such as exterior sheath designed to hold the microchannels, lack the structural integrity to retain attachment for the full desired time, as the biodegradation process weakens the sheath and the scaffolds become detached before full nerve recovery. This premature detachment of the implant can negatively impact patient outcomes, as a partially or fully detached scaffold may not properly align the neural tissue as it regrows.

The tissue repair scaffolds provided herein overcome these deficiencies through use of alignment markers on the exterior of the device, microchannel end point markers disposed on the exterior of the device, and/or optimized sheath thickness for enhanced stability after implantation.

Alignment Markers

In some aspects, provided herein, are markers disposed on a sheath which provide information to an installing surgeon about the alignment of one or more microchannels on the interior of the sheath. In some instances, the sheath is made from an opaque material which conceals the one or more microchannels on the interior of the device from the installing surgeon. In some embodiments, the one or more microchannels disposed within the interior of the device require proper alignment (e.g., a linear channel or a plurality of parallel linear channels) for optimal facilitation of nerve and/or axon growth through the microchannel. If the microchannel geometry becomes altered during installation, regrowing nerves or axons may become misaligned and prevent proper connectivity to desired tissues, such as peripheral organs (e.g., muscles). During handling and installation of such devices, the scaffolds may become twisted and thus alter the linear geometry of the interior microchannels. If a device is installed without proper alignment, the nerve reconnection may be compromised and result in sub-optimal nerve regeneration. Thus, a feature on the exterior of the device which signifies when the interior microchannels are properly aligned enables a surgeon to readily recognize when the device is in the proper orientation and thereby improve patient outcomes.

In one aspect, provided herein, is a tissue repair scaffold comprising: a sheath having a first end and a second end; a microchannel disposed within an interior of the sheath, wherein the microchannel traverses the sheath from the first end to the second end; and a marker disposed on an exterior surface of the sheath. In some embodiments, the marker indicates when the sheath is in the proper orientation.

The marker disposed on the exterior of the device can be any suitable marker which signifies that the device is in proper alignment (e.g., that the device is not twisted and/or that the interior channel(s) are in proper orientation). In some embodiments, the marker is a linear feature disposed on an exterior surface of the sheath. In some embodiments, the linear feature has been applied to the device after manufacture, such as by a printing or painting the feature or carving or impressing a groove onto the exterior of the sheath. In some embodiments, the linear feature is a line placed on an exterior of the sheath, and the line may be colored to allow easy determination of the alignment by an installing surgeon or other user. In some embodiments, the linear feature comprises a groove structure disposed on the exterior of the sheath. In some embodiments, the feature chosen is clearly visible during installation, such as by the naked eye on the exterior of the device or when viewed under a microscope or other visual enhancement device used during the installation process.

In some embodiments, the marker disposed on the exterior of the sheath runs substantially from the first end to the second end. In some embodiments, the marker comprises a feature which runs across at least about 50%, 60%, 70%, 80%, 90%, or 95% of the length of the device. In some embodiments, the length of the device is measured as the full sheath length, which can include an overhang structure which extends beyond the interior microchannel(s) (e.g., an overhang structure to allow for suturing the device to a nerve stump). In some embodiments, the length (e.g., the distance from the first end to the second end) does not encompass any overhang structure and is measured as the length of the interior microchannel(s).

In some embodiments, the marker is not an uninterrupted feature that proceeds across the entire or substantially entire length of the device. In some embodiments, the feature may comprise gaps, thus forming a dotted, dashed, or other similar alignment marker. In some embodiments, the sheath comprises two features disposed at opposite ends of the device with substantially no intervening marker, yet which still allows ready determination of proper alignment (e.g., on a 3 cm tissue repair scaffold device, two features running a shorter length such as 0.5 cm at opposite ends of the device with an interrupted gap between the features).

In some embodiments, the tissue repair scaffold comprises more than one marker on the exterior of the device. In some embodiments, the tissue repair scaffold comprises at least 2, 3, 4, 5, 6, 7, 8, 9, 10, or more markers. In some embodiments, the tissue repair scaffold comprises at least 2, 3, or 4 markers. In some embodiments, the tissue repair scaffold comprises 1, 2, 3, 4, 5, 6, 7, 8, 9, or 10 markers. In some embodiments, the tissue repair scaffold comprises 4 markers.

In some embodiments, the tissue repair scaffold comprises multiple markers which allow a user to ascertain if the device is in proper alignment when viewing the device from any orientation external to the device. In some embodiments, the sheath comprises markers disposed about a circumference of the sheath. In some embodiments, each of the markers is about evenly dispersed about the circumference of the sheath. In some embodiments, about evenly dispersed about the circumference of the sheath means that the markers are disposed evenly within a set tolerance, such as within about 25%, 20%, 15%, 10%, or 5% of evenly dispersed. In some embodiments, the tolerance is measured in degrees, such as each marker being within about 30 degrees, 25 degrees, 20 degrees, 15 degrees, 10 degrees, or 5 degrees of even dispersion.

In some embodiments, the marker(s) is/are substantially parallel with the microchannel. In some embodiments, wherein the device comprises a plurality of microchannels, the marker(s) is/are substantially parallel with a majority of the plurality of microchannels. In some embodiments, the marker(s) is/are substantially parallel with each of the plurality of microchannels. In some embodiments, the marker(s) is/are parallel with the microchannel. In some embodiments, substantially parallel means that the marker and the microchannel are within a set tolerance of parallel, such as within 10, 9, 8, 7, 6, 5, 4, 3, 2, or 1 degree of parallel.

In some embodiments, the marker comprises one or more grooves in the exterior of the sheath. The grooves can be added to the sheath during the manufacturing process of the scaffold (e.g., through a molding or casting process) or can be added to an otherwise completed scaffold (e.g., by embossing, boring, or etching). In some embodiments, the groove has a depth and width selected such that it can be readily seen either by the naked eye or when viewed under a microscope or other visual enhancement device used during the installation process. In some embodiments, the groove has a depth of about 1 microns to about 200 microns. In some embodiments, the groove has a depth of about 1 microns, about 5 microns, about 10 microns, about 50 microns, about 100 microns, about 150 microns, or about 200 microns. In some embodiments, the groove has a depth of at least about 1 microns, about 5 microns, about 10 microns, about 50 microns, about 100 microns, or about 150 microns. In some embodiments, the groove has a depth of at most about 5 microns, about 10 microns, about 50 microns, about 100 microns, about 150 microns, or about 200 microns. In some embodiments, the groove has a width of from about 1 to about 200 microns. In some embodiments, the groove has a width of about 1 microns, about 5 microns, about 10 microns, about 50 microns, about 100 microns, about 150 microns, or about 200 microns. In some embodiments, the groove has a width of at least about 1 microns, about 5 microns, about 10 microns, about 50 microns, about 100 microns, or about 150 microns. In some embodiments, the groove has a width of at most about 5 microns, about 10 microns, about 50 microns, about 100 microns, about 150 microns, or about 200 microns. In some embodiments, the shape of the groove is round or angular.

Microchannel Termination Markers

In some aspects, provided herein, are tissue repair scaffolds with markers disposed on an exterior surface of the sheath which demarcate the terminal point of one or more microchannels disposed within the sheath. The sheath comprises an overhang structure which extends beyond the point of demarcation by the marker. In some embodiments, the overhang structure is configured to be used to suture the scaffold to the tissue, such as a nerve stump of a damaged or severed nerve. The markers allow a user, such as a surgeon tasked with implanting the tissue repair scaffold into a subject, to ascertain where the microchannels on the interior of the device are located while viewing the exterior of the device, the sheath of which is opaque and obscures the interior microchannels. With this information, the surgeon can implant the device by suturing the overhang to a tissue site (e.g., a nerve stump) without piercing the interior microchannel(s) and causing damage to the device. If the microchannel(s) is compromised or damaged in this way, nerves or axons which grow through the device can become misaligned and meander through the orifice caused by the puncturing of the microchannel, thus compromising the ability of the scaffold to properly repair the nerve. A tissue repair scaffold which does not comprise the microchannel termination markers provided herein is thus more difficult for the surgeon to implant, requiring the surgeon to make many adjustments to the device to ascertain where on the device he or she can safely suture, and will be more likely to result in a scaffold which has been compromised prior to or during implantation, which could escape detection and lead to unfavorable patient outcomes.

In one aspect, provided herein, is a tissue repair scaffold comprising: a microchannel having a first end and a second end; and a sheath comprising: the microchannel disposed within an interior of the sheath, an overhang structure, wherein the overhang structure extends beyond the first end or the second of the microchannel, and a marker on an exterior of the sheath at a location that corresponds with the first end or the send end of the microchannel.

In some embodiments, the marker denotes a point on the exterior of the device that is the same point at which the microchannel(s) on the interior of the device terminate. Thus, the surgeon, upon seeing the marker, can immediately ascertain where the overhang begins and the microchannels end, and thus where he or she can safely suture the device to tissue of a subject (e.g., a nerve stump). In some embodiments, the marker feature disposed on an exterior surface of the sheath which runs circumferentially around the sheath. In some embodiments, the marker feature is a ring which runs at least partially circumferentially around the sheath. In some embodiments, the marker has been applied to the device after manufacture, such as by a printing or painting the marker or carving or embossing a groove onto the exterior of the sheath. In some embodiments, the microchannel termination marker is a line placed on an exterior of the sheath, and the line may be colored to allow easy determination of the alignment by an installing surgeon or other user. In some embodiments, the marker comprises a groove structure disposed on the exterior of the sheath. In some embodiments, the marker chosen is clearly visible during installation, such as by the naked eye on the exterior of the device or when viewed under a microscope or other visual enhancement device used during the installation process.

In some embodiments, the marker is positioned to circumferentially encompass the exterior of the sheath. In some embodiments, the marker is not a continuous feature around the circumference of the sheath, but rather is an interrupted feature, such as a dashed or dotted line or a series of small grooves disposed on the exterior of the sheath.

In some embodiments, the tissue repair scaffold comprises two microchannel end point markers. In some embodiments, the two microchannel end point markers are positioned at opposite ends of the scaffold.

In some embodiments, the microchannel termination marker comprises a groove disposed on the exterior of the sheath. The grooves can be added to the sheath during the manufacturing process of the scaffold (e.g., through a molding or casting process) or can be added to an otherwise completed scaffold (e.g., by embossing, boring, or etching). In some embodiments, the groove has a depth and width selected such that it can be readily seen either by the naked eye or when viewed under a microscope or other visual enhancement device used during the installation process. In some embodiments, the groove has a depth of about 1 microns to about 200 microns. In some embodiments, the groove has a depth of about 1 microns, about 5 microns, about 10 microns, about 50 microns, about 100 microns, about 150 microns, or about 200 microns. In some embodiments, the groove has a depth of at least about 1 microns, about 5 microns, about 10 microns, about 50 microns, about 100 microns, or about 150 microns. In some embodiments, the groove has a depth of at most about 5 microns, about 10 microns, about 50 microns, about 100 microns, about 150 microns, or about 200 microns. In some embodiments, the groove has a width of from about 1 microns to about 5 mm. In some embodiments, the groove has a width of at least about 1 microns, at least about 5 microns, at least about 10 microns, at least about 50 microns, at least about 100 microns, at least about 150 microns, or at least about 200 microns. In some embodiments, the groove has a width of at least about 0.25 mm, at least about 0.5 mm, at least about 1 mm, or at least about 1.5 mm. In some embodiments, the groove has a width of at most 5 mm, at most about 4 mm, at most about 3 mm, or at most about 2.5 mm.

In some embodiments, the overhang structure extends the sheath beyond the first end or the second end of the microchannels. In some embodiments, sheath comprises two overhang structures which extend the sheath beyond both the first end and the second end.

In some embodiments, the overhang structure extends beyond a terminal point of the one or more microchannels. The overhang structure should be a sufficient length to allow a surgeon to suture the overhang structure to a nerve stump or other tissue. In some embodiments, the overhang structure has a length of at least about 0.25 mm, at least about 0.4 mm, at least about 0.5 mm, at least about 0.6 mm, at least about 0.7 mm, at least about 0.8 mm, at least 0.9 mm, at least about 1 mm, at least about 1.1 mm, at least about 1.2 mm, at least about 1.3 mm, at least about 1.4 mm, at least about 1.5 mm, at least about 1.75 mm, or at least about 2 mm. In some embodiments, the overhang structure has a length of at least about 0.25 mm. In some embodiments, the overhang structure has a length of from about 0.25 mm to about 2 mm, about 0.4 mm to about 2 mm, about 0.5 to about 2 mm, about 0.25 mm to about 1.75 mm, about 0.25 mm to about 1.5 mm, about 0.5 mm to about 2 mm, or about 0.5 mm to about 1.5 mm. In some embodiments, the overhang structure has a length of about 0.5, 0.6, 0.7, 0.8, 0.9, 1.0, 1.1, 1.2, 1.3, 1.4, 1.5, 1.6, 1.7, 1.8, 1.9, or 2.0 mm.

The overhang structure should be a sufficient thickness to allow a surgeon to suture the overhang structure such that the overhang structure does not tear after installation. In some embodiments, the overhang structure has a thickness of about 100 microns to about 700 microns. In some embodiments, the overhang structure has a thickness of about 100 microns to about 200 microns, about 100 microns to about 300 microns, about 100 microns to about 400 microns, about 100 microns to about 500 microns, about 100 microns to about 600 microns, about 100 microns to about 700 microns, about 200 microns to about 300 microns, about 200 microns to about 400 microns, about 200 microns to about 500 microns, about 200 microns to about 600 microns, about 200 microns to about 700 microns, about 300 microns to about 400 microns, about 300 microns to about 500 microns, about 300 microns to about 600 microns, about 300 microns to about 700 microns, about 400 microns to about 500 microns, about 400 microns to about 600 microns, about 400 microns to about 700 microns, about 500 microns to about 600 microns, about 500 microns to about 700 microns, or about 600 microns to about 700 microns. In some embodiments, the overhang structure has a thickness of about 100 microns, about 200 microns, about 300 microns, about 400 microns, about 500 microns, about 600 microns, or about 700 microns. In some embodiments, the overhang structure has a thickness of at least about 100 microns, about 200 microns, about 300 microns, about 400 microns, about 500 microns, or about 600 microns. In some embodiments, the overhang structure has a thickness of at most about 200 microns, about 300 microns, about 400 microns, about 500 microns, about 600 microns, or about 700 microns.

The overhang structure may be made of any material of which the sheath may be made. In some embodiments, the overhang structure is made of the same material of which the sheath is made.

Improved Sheath Thickness

In some aspects, provided herein, are tissue repair scaffolds which have an optimized thickness of the sheath for stability and durability after implantation. In some embodiments, during installation into a subject, the tissue repair scaffold is sutured into a patient at the desired location. After installation, the tissue repair scaffold must remain stable for a period of time sufficient to allow the tissue to regenerate through the one or more microchannels. Thus, the sheath of the tissue repair scaffold must combine the features of durability during installation and stability thereafter. However, in some embodiments, it is undesirable for the tissue repair scaffold to remain for too long a period of time, as it is beneficial that the scaffold eventually biodegrades. Failure of the tissue repair scaffold to meet all of these criteria of stability and durability can negatively impact patient outcomes, such as detachment of the implant from the tissue (thereby preventing the tissue from reconnecting during regeneration). As the sheath is the portion of the tissue repair scaffold that makes and retains the connection of the device to the tissue of the subject, optimizing the properties of the sheath (such as composition and thickness) is of critical importance.

In one aspect, provided herein, is a tissue repair scaffold comprising: a sheath having a first end and a second end, wherein the sheath has a thickness of at least about 250 microns; and a microchannel disposed within an interior of the sheath, wherein the microchannels traverse the sheath from the first end to the second end. In some embodiments, the sheath optionally comprises an overhang structure which extends beyond the first end or the second end.

In one aspect, provided herein, is a tissue repair scaffold comprising: a sheath having a first end and a second end, wherein the sheath has a thickness of at least about 250 microns; and a plurality of microchannels disposed within an interior of the sheath, wherein the microchannels traverse the sheath from the first end to the second end. In some embodiments, the sheath optionally comprises an overhang structure which extends beyond the first end or the second end.

In some embodiments, the sheath has a thickness of about 250 microns to about 700 microns. In some embodiments, the sheath has a thickness of about 250 microns to about 300 microns, about 250 microns to about 350 microns, about 250 microns to about 400 microns, about 250 microns to about 450 microns, about 250 microns to about 500 microns, about 250 microns to about 550 microns, about 250 microns to about 600 microns, about 250 microns to about 650 microns, about 250 microns to about 700 microns, about 300 microns to about 350 microns, about 300 microns to about 400 microns, about 300 microns to about 450 microns, about 300 microns to about 500 microns, about 300 microns to about 550 microns, about 300 microns to about 600 microns, about 300 microns to about 650 microns, about 300 microns to about 700 microns, about 350 microns to about 400 microns, about 350 microns to about 450 microns, about 350 microns to about 500 microns, about 350 microns to about 550 microns, about 350 microns to about 600 microns, about 350 microns to about 650 microns, about 350 microns to about 700 microns, about 400 microns to about 450 microns, about 400 microns to about 500 microns, about 400 microns to about 550 microns, about 400 microns to about 600 microns, about 400 microns to about 650 microns, about 400 microns to about 700 microns, about 450 microns to about 500 microns, about 450 microns to about 550 microns, about 450 microns to about 600 microns, about 450 microns to about 650 microns, about 450 microns to about 700 microns, about 500 microns to about 550 microns, about 500 microns to about 600 microns, about 500 microns to about 650 microns, about 500 microns to about 700 microns, about 550 microns to about 600 microns, about 550 microns to about 650 microns, about 550 microns to about 700 microns, about 600 microns to about 650 microns, about 600 microns to about 700 microns, or about 650 microns to about 700 microns. In some embodiments, the sheath has a thickness of about 250 microns, about 300 microns, about 350 microns, about 400 microns, about 450 microns, about 500 microns, about 550 microns, about 600 microns, about 650 microns, or about 700 microns. In some embodiments, the sheath has a thickness of at least about 250 microns, about 300 microns, about 350 microns, about 400 microns, about 450 microns, about 500 microns, about 550 microns, about 600 microns, or about 650 microns. In some embodiments, the sheath has a thickness of at most about 300 microns, about 350 microns, about 400 microns, about 450 microns, about 500 microns, about 550 microns, about 600 microns, about 650 microns, or about 700 microns.

In some embodiments, the sheath is made of a biodegradable polymer. In some embodiments, the biodegradable polymer is selected from polyethylene glycol, polyethylene glycol diacrylate, gelatin, methacrylated gelatin, collagen, methacrylated collagen, polylactic acid, poly glycolic acid, poly(lactic-co-glycolic acid), polycaprolactone, and acrylated polycaprolactone, or any combination thereof. In some embodiments, the biodegradable polymer is selected from polyethylene glycol, polyethylene glycol diacrylate, gelatin, methacrylated gelatin, collagen, methacrylated collagen, poly(lactic-co-glycolic acid), polycaprolactone, and acrylated polycaprolactone, or any combination thereof. In some embodiments, the biodegradable polymer is selected from poly(lactic-co-glycolic acid), polycaprolactone, and acrylated polycaprolactone, or any combination thereof. In some embodiments, the biodegradable polymer is selected from poly(lactic-co-glycolic acid) and polycaprolactone, or a combination thereof. In some embodiments, the biodegradable polymer is poly(lactic-co-glycolic acid). In some embodiments, the biodegradable polymer is polycaprolactone.

In some embodiments, the sheath is made of polycaprolactone and has a thickness of at least about 250 microns, at least about 300 microns, at least about 350 microns, at least about 400 microns, at least about 450 microns, or at least about 500 microns. In some embodiments, the sheath is made of polycaprolactone and has a thickness of from about 250 microns to about 700 microns, about 250 microns to about 650 microns, about 250 microns to about 600 microns, about 250 microns to about 550 microns, about 300 microns to about 700 microns, about 300 microns to about 650 microns, about 300 microns to about 600 microns, about 300 microns to about 550 microns, about 350 microns to about 700 microns, about 350 microns to about 650 microns, about 350 microns to about 600 microns, about 350 microns to about 550 microns, about 400 microns to about 700 microns, about 400 microns to about 650 microns, about 400 microns to about 600 microns, or about 400 microns to about 550 microns. In some embodiments, the sheath is made of polycaprolactone and has a thickness of about 250 microns, about 300 microns, about 350 microns, about 400 microns, about 450 microns, about 500 microns, about 550 microns, about 600 microns, about 650 microns, or about 700 microns.

In some embodiments, the sheath comprises a plurality of pores. In some embodiments, the plurality of pores have an average particle size and particle size distribution such that the pores do not create holes within the sheath (e.g., no “line of sight” porosity within the walls). Thus, in some embodiments, the average pore size is selected to be significantly smaller than the sheath thickness. In some embodiments, the plurality of pores have an average particle size ranging from about 1 microns to about 100 microns. In some embodiments, the plurality of pores have an average particle size from about 1 microns to about 50 microns. In some embodiments, the plurality of pores have an average particle size of about 1 micron to about 50 microns. In some embodiments, the plurality of pores have an average particle size of about 1 microns to about 5 microns, about 1 microns to about 10 microns, about 1 microns to about 20 microns, about 1 microns to about 30 microns, about 1 microns to about 40 microns, about 1 microns to about 50 microns, about 5 microns to about 10 microns, about 5 microns to about 20 microns, about 5 microns to about 30 microns, about 5 microns to about 40 microns, about 5 microns to about 50 microns, about 10 microns to about 20 microns, about 10 microns to about 30 microns, about 10 microns to about 40 microns, about 10 microns to about 50 microns, about 20 microns to about 30 microns, about 20 microns to about 40 microns, about 20 microns to about 50 microns, about 30 microns to about 40 microns, about 30 microns to about 50 microns, or about 40 microns to about 50 microns. In some embodiments, the plurality of pores have an average particle size of about 1 microns, about 5 microns, about 10 microns, about 20 microns, about 30 microns, about 40 microns, or about 50 microns. In some embodiments, the plurality of pores have an average particle size of at least about 1 microns, about 5 microns, about 10 microns, about 20 microns, about 30 microns, or about 40 microns. In some embodiments, the plurality of pores have an average particle size of at most about 5 microns, about 10 microns, about 20 microns, about 30 microns, about 40 microns, or about 50 microns.

In some embodiments, the sheath has a high degree of porosity. In some embodiments, the sheath has a porosity of about 40% vol to about 75% vol. In some embodiments, the sheath has a porosity of about 40% vol to about 50% vol, about 40% vol to about 60% vol, about 40% vol to about 65% vol, about 40% vol to about 70% vol, about 40% vol to about 75% vol, about 50% vol to about 60% vol, about 50% vol to about 65% vol, about 50% vol to about 70% vol, about 50% vol to about 75% vol, about 60% vol to about 65% vol, about 60% vol to about 70% vol, about 60% vol to about 75% vol, about 65% vol to about 70% vol, about 65% vol to about 75% vol, or about 70% vol to about 75% vol. In some embodiments, the sheath has a porosity of about 40% vol, about 50% vol, about 60% vol, about 65% vol, about 70% vol, or about 75% vol. In some embodiments, the sheath has a porosity of at least about 40% vol, about 50% vol, about 60% vol, about 65% vol, or about 70% vol. In some embodiments, the sheath has a porosity of at most about 50% vol, about 60% vol, about 65% vol, about 70% vol, or about 75% vol.

In some embodiments, the sheath is stable for a sufficient period of time to allow for the nerve to sufficiently regrow. In some embodiments, the sheath is stable after implantation. In some embodiments, the sheath is stable after implantation in the subject for a period of at least 90 days, at least 100 days, at least 110 days, at least 120 days, at least 150 days, at least 180 days, at least 210 days, at least 240 days, at least 270 days, at least 300 days, at least 330 days, or at least 365 days. In some embodiments, the stability is measured as a percentage of substantially identical scaffolds which remain attached to a nerve segment in a model animal (e.g., rabbit) after the period of time (e.g., at least 80%, at least 85%, at least 90%, at least 95%, or 100% of scaffolds tested remain attached to the nerve segment of the model animal after the period of time).

Tissue Repair Scaffolds

Provided herein are tissue repair scaffolds which have the improved features for implantation provided herein. In some embodiments, the tissue repair scaffolds are specialized for the repair of nerves. In some embodiments, the tissue repair scaffolds comprise a sheath and one or more microchannels disposed within the sheath.

In some embodiments, the sheath comprises one or more of the improved features for implantation provided herein, such as the alignment markets, the microchannel end point markers, and/or the increased sheath thickness. In some embodiments, the sheath comprises one of these features. In some embodiments, the sheath comprises any combination of two of these features. In some embodiments, the sheath comprises each of these three features.

The sheaths provided herein can be made from a variety of materials which are suitable for implantation into a subject. In some embodiments, the sheath is made from a biodegradable material. In some embodiments, the sheath is made from a biodegradable polymer. In some embodiments, the sheath is made from at least 50%, at least 60%, at least 70%, at least 80%, at least 90%, at least 95%, or at least 98% of the biodegradable polymer. In some embodiments, the biodegradable polymer is selected from polyethylene glycol, polyethylene glycol diacrylate, gelatin, methacrylated gelatin, collagen, methacrylated collagen, polylactic acid, poly glycolic acid, poly(lactic-co-glycolic acid), polycaprolactone, and acrylated polycaprolactone, or any combination thereof. In some embodiments, the biodegradable polymer is selected from polyethylene glycol, polyethylene glycol diacrylate, gelatin, methacrylated gelatin, collagen, methacrylated collagen, poly(lactic-co-glycolic acid), polycaprolactone, and acrylated polycaprolactone, or any combination thereof. In some embodiments, the biodegradable polymer is selected from poly(lactic-co-glycolic acid), polycaprolactone, and acrylated polycaprolactone, or any combination thereof. In some embodiments, the biodegradable polymer is selected from poly(lactic-co-glycolic acid) and polycaprolactone, or a combination thereof. In some embodiments, the biodegradable polymer is poly(lactic-co-glycolic acid). In some embodiments, the biodegradable polymer is polycaprolactone.

In some embodiments, the sheath comprises an improved sheath thickness as set forth herein. In some embodiments, the sheath has a different thickness (e.g., a thinner sheath thickness than those provided elsewhere herein). While such sheaths may lack the advantages of the improved sheath thicknesses provided herein, in certain contexts they may provide other advantages.

In some embodiments, the sheath has a thickness of about 50 microns to about 500 microns. In some embodiments, the sheath has a thickness of about 50 microns to about 75 microns, about 50 microns to about 100 microns, about 50 microns to about 150 microns, about 50 microns to about 200 microns, about 50 microns to about 250 microns, about 50 microns to about 500 microns, about 75 microns to about 100 microns, about 75 microns to about 150 microns, about 75 microns to about 200 microns, about 75 microns to about 250 microns, about 75 microns to about 500 microns, about 100 microns to about 150 microns, about 100 microns to about 200 microns, about 100 microns to about 250 microns, about 100 microns to about 500 microns, about 150 microns to about 200 microns, about 150 microns to about 250 microns, about 150 microns to about 500 microns, about 200 microns to about 250 microns, about 200 microns to about 500 microns, or about 250 microns to about 500 microns. In some embodiments, the sheath has a thickness of about 50 microns, about 75 microns, about 100 microns, about 150 microns, about 200 microns, about 250 microns, or about 500 microns. In some embodiments, the sheath has a thickness of at least about 50 microns, about 75 microns, about 100 microns, about 150 microns, about 200 microns, or about 250 microns. In some embodiments, the sheath has a thickness of at most about 75 microns, about 100 microns, about 150 microns, about 200 microns, about 250 microns, or about 500 microns.

The sheath has an interior diameter sufficient to accommodate a desired number of microchannels having a desired diameter. The size of the sheath interior diameter will depend on a number of factors specific to the intended use of the tissue repair scaffold, including the type of tissue to be repaired and number of axons or nerves which much be connected (and thus the number microchannels).

In some embodiments, the sheath has an inner diameter of about 1 mm to about 10 mm. In some embodiments, the sheath has an inner diameter of about 1 mm to about 1.5 mm, about 1 mm to about 2 mm, about 1 mm to about 2.5 mm, about 1 mm to about 3 mm, about 1 mm to about 3.5 mm, about 1 mm to about 4 mm, about 1 mm to about 5 mm, about 1 mm to about 7.5 mm, about 1 mm to about 10 mm, about 1.5 mm to about 2 mm, about 1.5 mm to about 2.5 mm, about 1.5 mm to about 3 mm, about 1.5 mm to about 3.5 mm, about 1.5 mm to about 4 mm, about 1.5 mm to about 5 mm, about 1.5 mm to about 7.5 mm, about 1.5 mm to about 10 mm, about 2 mm to about 2.5 mm, about 2 mm to about 3 mm, about 2 mm to about 3.5 mm, about 2 mm to about 4 mm, about 2 mm to about 5 mm, about 2 mm to about 7.5 mm, about 2 mm to about 10 mm, about 2.5 mm to about 3 mm, about 2.5 mm to about 3.5 mm, about 2.5 mm to about 4 mm, about 2.5 mm to about 5 mm, about 2.5 mm to about 7.5 mm, or about 2.5 mm to about 10 mm. In some embodiments, the sheath has an inner diameter of about 1 mm, about 1.5 mm, about 2 mm, about 2.5 mm, about 3 mm, about 3.5 mm, about 4 mm, about 5 mm, about 7.5 mm, or about 10 mm. In some embodiments, the sheath has an inner diameter of at least about 1 mm, about 1.5 mm, about 2 mm, or about 2.5 mm. In some embodiments, the sheath has an inner diameter of at most about 5 mm, about 7.5 mm, or about 10 mm. In some embodiments, the sheath has an internal diameter of about 3 mm.

In some embodiments, the device comprises an overhang structure. The overhang structure may be used to suture the device to the nerve stump or other relevant tissue, thereby placing the tissue at the proper location and orientation to grow through the microchannel(s) of the device and regenerate the tissue. In some embodiments, the overhang structure extends the sheath beyond a first end or a second end of the microchannels. In some embodiments, sheath comprises two overhang structures which extend the sheath beyond both the first end and the second end.

In some embodiments, the overhang structure extends beyond a terminal point of the one or more microchannels. The overhang structure should be a sufficient length to allow a surgeon to suture the overhang structure to a nerve stump or other tissue. In some embodiments, the overhang structure has a length of at least about 0.25 mm, at least about 0.4 mm, at least about 0.5 mm, at least about 0.6 mm, at least about 0.7 mm, at least about 0.8 mm, at least 0.9 mm, at least about 1 mm, at least about 1.1 mm, at least about 1.2 mm, at least about 1.3 mm, at least about 1.4 mm, at least about 1.5 mm, at least about 1.75 mm, or at least about 2 mm. In some embodiments, the overhang structure has a length of at least about 0.25 mm. In some embodiments, the overhang structure has a length of from about 0.25 mm to about 2 mm, about 0.4 mm to about 2 mm, about 0.5 to about 2 mm, about 0.25 mm to about 1.75 mm, about 0.25 mm to about 1.5 mm, about 0.5 mm to about 2 mm, or about 0.5 mm to about 1.5 mm. In some embodiments, the overhang structure has a length of about 0.5, 0.6, 0.7, 0.8, 0.9, 1.0, 1.1, 1.2, 1.3, 1.4, 1.5, 1.6, 1.7, 1.8, 1.9, or 2.0 mm.

The overhang structure should be a sufficient thickness to allow a surgeon to suture the overhang structure such that the overhang structure does not tear after installation. In some embodiments, the overhang structure has substantially the same thickness as the sheath. In some embodiments, the overhang structure has a thickness of about 100 microns to about 700 microns. In some embodiments, the overhang structure has a thickness of about 100 microns to about 200 microns, about 100 microns to about 300 microns, about 100 microns to about 400 microns, about 100 microns to about 500 microns, about 100 microns to about 600 microns, about 100 microns to about 700 microns, about 200 microns to about 300 microns, about 200 microns to about 400 microns, about 200 microns to about 500 microns, about 200 microns to about 600 microns, about 200 microns to about 700 microns, about 300 microns to about 400 microns, about 300 microns to about 500 microns, about 300 microns to about 600 microns, about 300 microns to about 700 microns, about 400 microns to about 500 microns, about 400 microns to about 600 microns, about 400 microns to about 700 microns, about 500 microns to about 600 microns, about 500 microns to about 700 microns, or about 600 microns to about 700 microns. In some embodiments, the overhang structure has a thickness of about 100 microns, about 200 microns, about 300 microns, about 400 microns, about 500 microns, about 600 microns, or about 700 microns. In some embodiments, the overhang structure has a thickness of at least about 100 microns, about 200 microns, about 300 microns, about 400 microns, about 500 microns, or about 600 microns. In some embodiments, the overhang structure has a thickness of at most about 200 microns, about 300 microns, about 400 microns, about 500 microns, about 600 microns, or about 700 microns. In some embodiments, the overhang structure has a thickness of about 500 microns.

The overhang structure may be made of any material of which the sheath may be made. In some embodiments, the overhang structure is made of the same material of which the sheath is made. In some embodiments, the overhang structure is fabricated as part of the sheath.

The tissue repair scaffolds have one or more microchannels disposed within the sheath. In some embodiments, the tissue repair scaffolds comprise a single microchannel disposed within the sheath. In some embodiments, the tissue repair scaffold comprises a plurality of microchannels disposed within the sheath.

The microchannels may be made from any suitable biocompatible material. In some embodiments, the microchannels are made from the same material as the sheath. In some embodiments, the microchannels are made from a different material as the sheath.

In some embodiments, the microchannels are made from a biodegradable material. In some embodiments, the microchannels are made from a biodegradable polymer. In some embodiments, the microchannels are made from at least 50%, at least 60%, at least 70%, at least 80%, at least 90%, at least 95%, or at least 98% of the biodegradable polymer. In some embodiments, the biodegradable polymer is selected from polyethylene glycol, polyethylene glycol diacrylate, gelatin, methacrylated gelatin, collagen, methacrylated collagen, polylactic acid, poly glycolic acid, poly(lactic-co-glycolic acid), polycaprolactone, and acrylated polycaprolactone, or any combination thereof. In some embodiments, the biodegradable polymer is selected from polyethylene glycol, polyethylene glycol diacrylate, gelatin, methacrylated gelatin, collagen, methacrylated collagen, poly(lactic-co-glycolic acid), polycaprolactone, and acrylated polycaprolactone, or any combination thereof. In some embodiments, the biodegradable polymer is selected from poly(lactic-co-glycolic acid), polycaprolactone, and acrylated polycaprolactone, or any combination thereof. In some embodiments, the biodegradable polymer is selected from poly(lactic-co-glycolic acid) and polycaprolactone, or a combination thereof. In some embodiments, the biodegradable polymer is poly(lactic-co-glycolic acid). In some embodiments, the biodegradable polymer is polycaprolactone.

In some embodiments, the microchannels comprise a plurality of pores disposed within the walls. In some embodiments, the plurality of pores have an average particle size and particle size distribution such that the pores do not create holes within the microchannel walls (e.g., no “line of sight” porosity within the walls). Thus, in some embodiments, the average pore size is selected to be significantly smaller than the wall thickness (e.g., ½ the size or less). In some embodiments, the plurality of pores have an average particle size ranging from about 1 micron to about 100 microns. In some embodiments, the plurality of pores have an average particle size from about 1 microns to about 50 microns. In some embodiments, the plurality of pores have an average particle size of about 1 microns to about 50 microns. In some embodiments, the plurality of pores have an average particle size of about 1 microns to about 5 microns, about 1 microns to about 10 microns, about 1 microns to about 20 microns, about 1 microns to about 30 microns, about 1 microns to about 40 microns, about 1 microns to about 50 microns, about 5 microns to about 10 microns, about 5 microns to about 20 microns, about 5 microns to about 30 microns, about 5 microns to about 40 microns, about 5 microns to about 50 microns, about 10 microns to about 20 microns, about 10 microns to about 30 microns, about 10 microns to about 40 microns, about 10 microns to about 50 microns, about 20 microns to about 30 microns, about 20 microns to about 40 microns, about 20 microns to about 50 microns, about 30 microns to about 40 microns, about 30 microns to about 50 microns, or about 40 microns to about 50 microns. In some embodiments, the plurality of pores have an average particle size of about 1 microns, about 5 microns, about 10 microns, about 20 microns, about 30 microns, about 40 microns, or about 50 microns. In some embodiments, the plurality of pores have an average particle size of at least about 1 microns, about 5 microns, about 10 microns, about 20 microns, about 30 microns, or about 40 microns. In some embodiments, the plurality of pores have an average particle size of at most about 5 microns, about 10 microns, about 20 microns, about 30 microns, about 40 microns, or about 50 microns.

In some embodiments, the walls of the microchannels have a high degree of porosity. In some embodiments, the walls of the microchannels have a porosity of about 40% vol to about 75% vol. In some embodiments, the walls of the microchannels have a porosity of about 40% vol to about 50% vol, about 40% vol to about 60% vol, about 40% vol to about 65% vol, about 40% vol to about 70% vol, about 40% vol to about 75% vol, about 50% vol to about 60% vol, about 50% vol to about 65% vol, about 50% vol to about 70% vol, about 50% vol to about 75% vol, about 60% vol to about 65% vol, about 60% vol to about 70% vol, about 60% vol to about 75% vol, about 65% vol to about 70% vol, about 65% vol to about 75% vol, or about 70% vol to about 75% vol. In some embodiments, the walls of the microchannels have a porosity of about 40% vol, about 50% vol, about 60% vol, about 65% vol, about 70% vol, or about 75% vol. In some embodiments, the walls of the microchannels have a porosity of at least about 40% vol, about 50% vol, about 60% vol, about 65% vol, or about 70% vol. In some embodiments, the walls of the microchannels have a porosity of at most about 50% vol, about 60% vol, about 65% vol, about 70% vol, or about 75% vol.

In some embodiments, the walls of the microchannels are made from a material that is inherently porous to diffusion of nutrients through the barrier, yet do not comprise pores (e.g., a hydrogel material. In some embodiments, the walls of the microchannels comprise hydrogels.

In some embodiments, the microchannels have walls that the plurality of channels. In some embodiments, the walls of the microchannel walls have a thickness of about 10 microns to about 100 microns. In some embodiments, the walls of the microchannel walls have a thickness of about 10 microns to about 20 microns, about 10 microns to about 30 microns, about 10 microns to about 40 microns, about 10 microns to about 60 microns, about 10 microns to about 80 microns, about 10 microns to about 100 microns, about 20 microns to about 30 microns, about 20 microns to about 40 microns, about 20 microns to about 60 microns, about 20 microns to about 80 microns, about 20 microns to about 100 microns, about 30 microns to about 40 microns, about 30 microns to about 60 microns, about 30 microns to about 80 microns, about 30 microns to about 100 microns, about 40 microns to about 60 microns, about 40 microns to about 80 microns, about 40 microns to about 100 microns, about 60 microns to about 80 microns, about 60 microns to about 100 microns, or about 80 microns to about 100 microns. In some embodiments, the walls of the microchannel walls have a thickness of about 10 microns, about 20 microns, about 30 microns, about 40 microns, about 60 microns, about 80 microns, or about 100 microns. In some embodiments, the walls of the microchannel walls have a thickness of at least about 10 microns, about 20 microns, about 30 microns, about 40 microns, about 60 microns, or about 80 microns. In some embodiments, the walls of the microchannel walls have a thickness of at most about 20 microns, about 30 microns, about 40 microns, about 60 microns, about 80 microns, or about 100 microns.

The microchannels should have an internal diameter which is optimized for the use for which it is intended, which will depend on the application. In some embodiments, the internal diameter is optimized for a particular nerve of interest (e.g., a spinal nerve or a peripheral nerve). In some embodiments, the microchannels have an inner dimeter of about 50 microns to about 500 microns. In some embodiments, microchannels have an inner diameter of about 50 microns to about 300 microns. In some embodiments, microchannels have an inner diameter of about 50 microns to about 100 microns, about 50 microns to about 150 microns, about 50 microns to about 175 microns, about 50 microns to about 200 microns, about 50 microns to about 225 microns, about 50 microns to about 250 microns, about 50 microns to about 300 microns, about 100 microns to about 150 microns, about 100 microns to about 175 microns, about 100 microns to about 200 microns, about 100 microns to about 225 microns, about 100 microns to about 250 microns, about 100 microns to about 300 microns, about 150 microns to about 175 microns, about 150 microns to about 200 microns, about 150 microns to about 225 microns, about 150 microns to about 250 microns, about 150 microns to about 300 microns, about 175 microns to about 200 microns, about 175 microns to about 225 microns, about 175 microns to about 250 microns, about 175 microns to about 300 microns, about 200 microns to about 225 microns, about 200 microns to about 250 microns, about 200 microns to about 300 microns, about 225 microns to about 250 microns, about 225 microns to about 300 microns, or about 250 microns to about 300 microns. In some embodiments, microchannels have an inner diameter of about 50 microns, about 100 microns, about 150 microns, about 175 microns, about 200 microns, about 225 microns, about 250 microns, or about 300 microns. In some embodiments, microchannels have an inner diameter of at least about 50 microns, about 100 microns, about 150 microns, about 175 microns, about 200 microns, about 225 microns, or about 250 microns. In some embodiments, microchannels have an inner diameter of at most about 100 microns, about 150 microns, about 175 microns, about 200 microns, about 225 microns, about 250 microns, or about 300 microns.

The microchannels can have any shape suitable for growing the desired tissue through the channel. In some embodiments, the microchannel shape is the cross-sectional shape of the channels. In some embodiments, the microchannels are hexagonal, round, triangular, rectangular, square, pentagonal, heptagonal, octagonal, nonagonal, decagonal, elliptical, or trapezoidal in shape. In some embodiments, the microchannels are hexagonal or round in shape. In some embodiments, the microchannels are hexagonal. In some embodiments, the microchannels are round.

In some embodiments, the tissue repair scaffolds comprise a plurality of microchannels. The number of microchannels of the device is selected based on the relevant tissue. In some embodiments, the tissue repair scaffold comprises about 2 to about 300 microchannels. In some embodiments, the tissue repair scaffold comprises about 2 to about 5, about 2 to about 7, about 2 to about 15, about 2 to about 30, about 2 to about 50, about 2 to about 100, about 2 to about 150, about 2 to about 200, about 2 to about 300, about 5 to about 7, about 5 to about 15, about 5 to about 30, about 5 to about 50, about 5 to about 100, about 5 to about 150, about 5 to about 200, about 5 to about 300, about 7 to about 15, about 7 to about 30, about 7 to about 50, about 7 to about 100, about 7 to about 150, about 7 to about 200, about 7 to about 300, about 15 to about 30, about 15 to about 50, about 15 to about 100, about 15 to about 150, about 15 to about 200, about 15 to about 300, about 30 to about 50, about 30 to about 100, about 30 to about 150, about 30 to about 200, about 30 to about 300, about 50 to about 100, about 50 to about 150, about 50 to about 200, about 50 to about 300, about 100 to about 150, about 100 to about 200, about 100 to about 300, about 150 to about 200, about 150 to about 300, or about 200 to about 300 microchannels. In some embodiments, the tissue repair scaffold comprises about 2, about 5, about 7, about 15, about 30, about 50, about 100, about 150, about 200, or about 300 microchannels. In some embodiments, the tissue repair scaffold comprises at least about 2, about 5, about 7, about 15, about 30, about 50, about 100, about 150, or about 200 microchannels. In some embodiments, the tissue repair scaffold comprises at most about 5, about 7, about 15, about 30, about 50, about 100, about 150, about 200, or about 300 microchannels.

In some embodiments, the tissue repair scaffold comprising a plurality of microchannels will have a desired microchannel density. The desired microchannel density will depend on the application for which the tissue repair scaffold is intended. The microchannel density may be varied in different embodiments, for example, the microchannel density may be greater than or equal to about 1 to less than or equal to about 300 microchannels/mm 2 in the scaffold. In certain variations, the microchannel density may be greater than or equal to about 10 to less than or equal to about 30 microchannels/mm². In another variation, the tissue scaffold may have a microchannel density of about 120 microchannels/mm². In some variations, the microchannel density is about 10 to about 300 microchannels/mm². In some variations, the microchannel density is about 10 to about 20, about 10 to about 30, about 10 to about 50, about 10 to about 100, about 10 to about 120, about 10 to about 150, about 10 to about 200, about 10 to about 300, about 20 to about 30, about 20 to about 50, about 20 to about 100, about 20 to about 120, about 20 to about 150, about 20 to about 200, about 20 to about 300, about 30 to about 50, about 30 to about 100, about 30 to about 120, about 30 to about 150, about 30 to about 200, about 30 to about 300, about 50 to about 100, about 50 to about 120, about 50 to about 150, about 50 to about 200, about 50 to about 300, about 100 to about 120, about 100 to about 150, about 100 to about 200, about 100 to about 300, about 120 to about 150, about 120 to about 200, about 120 to about 300, about 150 to about 200, about 150 to about 300, or about 200 to about 300 microchannels/mm². In some variations, the microchannel density is about 10, about 20, about 30, about 50, about 100, about 120, about 150, about 200, or about 300 microchannels/mm². In some variations, the microchannel density is at least about 10, about 20, about 30, about 50, about 100, about 120, about 150, or about 200 microchannels/mm². In some variations, the microchannel density is at most about 20, about 30, about 50, about 100, about 120, about 150, about 200, or about 300 microchannels/mm².

The scaffold can have any suitable length needed to treat a tissue injury, such as a nerve injury. In some embodiments, the scaffold can be from 0.5 mm to 10 cm in length. In some embodiments, the scaffold can be from 5 mm to 10 cm in length. In some embodiments, the scaffold can be up to 15 cm in length. In some embodiments, the length of the scaffold is about 0.5 cm to about 10 cm. In some embodiments, the length of the scaffold is about 0.5 cm to about 1 cm, about 0.5 cm to about 2 cm, about 0.5 cm to about 3 cm, about 0.5 cm to about 5 cm, about 0.5 cm to about 7 cm, about 0.5 cm to about 9 cm, about 0.5 cm to about 10 cm, about 1 cm to about 2 cm, about 1 cm to about 3 cm, about 1 cm to about 5 cm, about 1 cm to about 7 cm, about 1 cm to about 9 cm, about 1 cm to about 10 cm, about 2 cm to about 3 cm, about 2 cm to about 5 cm, about 2 cm to about 7 cm, about 2 cm to about 9 cm, about 2 cm to about 10 cm, about 3 cm to about 5 cm, about 3 cm to about 7 cm, about 3 cm to about 9 cm, about 3 cm to about 10 cm, about 5 cm to about 7 cm, about 5 cm to about 9 cm, about 5 cm to about 10 cm, about 7 cm to about 9 cm, about 7 cm to about 10 cm, or about 9 cm to about 10 cm. In some embodiments, the length of the scaffold is about 0.5 cm, about 1 cm, about 2 cm, about 3 cm, about 5 cm, about 7 cm, about 9 cm, or about 10 cm. In some embodiments, the length of the scaffold is at least about 0.5 cm, about 1 cm, about 2 cm, about 3 cm, about 5 cm, about 7 cm, or about 9 cm. In some embodiments, the length of the scaffold is at most about 1 cm, about 2 cm, about 3 cm, about 5 cm, about 7 cm, about 9 cm, or about 10 cm. In some embodiments, the length of the scaffold is from about 0.1 cm to about 15 cm. In some embodiments, the length of the scaffold is from about 5 cm to about 15 cm. In some embodiments, the length of the scaffold is at least 5 cm.

In some embodiments, the tissue repair scaffold is filled with one or more cells to stimulate tissue regeneration. In some embodiments, the cells are modified to express a growth factor or can be therapeutic in nature such as stem cells or Schwann cells.

A portion of nerve, such as a nerve end, of the subject may be damaged or severed, for example, a fully or partially lesioned nerve end caused by injury, disease, or surgery. In certain aspects, a portion of the nerve end may be surgically divided, sectioned, cut, and/or transected into one or more individual branches or fascicles that may be secured to a proximal end or distal end of the tissue scaffold. The one or more individual branches or fascicles of the nerve end may contact or be placed within one or more microchannels. The nerve end (or its individual branches or fascicles) can be secured via sutures, adhesives, or other known securing techniques to the proximal or distal ends of the sheath. Over a period of, for example, several months, the neural tissue originating from the nerve end can grow along the longitudinal axis of each microchannel and reinnervate any neural targets at the opposite end of the tissue scaffold. The tissue scaffolds according to various aspects of the present teachings thus facilitate neural tissue growth through the open central lumens of the plurality of microchannels from a first end to a second opposite end of the scaffold.

In other aspects, surfaces of the walls of microchannels may be coated with a biofunctional agent to promote cell growth, regeneration, differentiation, proliferation, and/or repair, for example. By “promoting” cell growth, cell proliferation, cell differentiation, cell repair, or cell regeneration, it is meant that a detectable increase occurs in either a rate or a measurable outcome of such processes occurs in the presence of the biofunctional agent as compared to a cell or organism's process in the absence of such a biofunctional agent, for example, conducting such processes naturally. By way of example, as appreciated by those of skill in the art, promoting cell growth in the presence of a biofunctional agent may increase a growth rate of target cells or increase a total cell count of the target cells, when compared to cell growth or cell count of the target cells in the absence of such a biofunctional agent.

In some embodiments, the biofunctional agent is one which promotes cell growth, cell adhesion, cell proliferation, cell differentiation, cell repair, and/or cell regeneration by increasing a measurable process result (e.g., measuring total cell counts for cell generation or cell regeneration, measuring the rates or qualitative outcome of cell proliferation, cell differentiation, or cell repair rates). Exemplary biofunctional agents include, but are not limited to fibronectin, keratin, laminin, collagen, a growth factor, and/or a stem cell-promoting factor. Exemplary growth factors include brain derived neurotrophic factor (BDNF), nerve growth factor, glial cell-derived neurotrophic factor (GDNF), and neurotrophin-3 (NT-3). In some embodiments, the growth factor is BDNF. In some embodiments, the growth factor is nerve growth factor. In some embodiments, the growth factor is GDNF. In some embodiments, the growth factor is NT-3.

Such a biofunctional agent may be introduced after the microchannels are formed, for example, by coating, infusing, or otherwise incorporating the biofunctional agent onto one of more surfaces (e.g., internal surface) of the microchannel wall. In certain aspects, a surface of the porous wall has a coating comprising a material for promoting growth of the neural tissue selected from the group consisting of: fibronectin, keratin, laminin, collagen, and combinations and equivalents thereof. In certain embodiments, the walls may be coated with fibronectin, which has been found after screening over a dozen compounds to be particularly advantageous with the biocompatible polymers forming the microchannel walls to optimize cell and axon attachment.

In some embodiments, the tissue repair scaffold is configured to facilitate growth of tissue through the microchannel(s). In some embodiments, the tissue repair scaffold is configured to rejoin severed nerve tissue within the microchannel(s). In some embodiments, the nerve tissue is a spinal nerve or a peripheral nerve. In some embodiments, the nerve tissue is a spinal nerve. In some embodiments, the nerve tissue is a peripheral nerve.

One advantage of the tissue scaffold design according to various aspects of the present disclosure is providing an overall open volume (e.g., open lumen volume, including the volume of open interstitial channels within sheath and open central lumen of microchannels) of greater than or equal to about 50 volume %, optionally greater than or equal to about 60 volume %, optionally greater than or equal to about 70 volume %, optionally greater than or equal to about 80 volume %, and in certain preferred aspects, optionally greater than or equal to about 90 open volume % of the overall scaffold volume. It should be noted that conventional scaffold designs were not able to achieve such high levels of open lumen volumes, which is believed to be particularly advantageous in supporting and promoting growth of healthy neural tissues having desirably high directional linearity and high signal fidelity.

While bioengineered scaffolds or implants support axon regeneration into spinal cord, or peripheral nerve, lesion sites, these technologies have been limited by foreign body responses at implantation sites, cumbersome production requirements, limitations in scaling to human-sized injuries and lack of biomimicry of the natural spinal cord or peripheral nerve. The implants and methods of using the implants described herein include structures which biomimic complex fascicular architecture of a spinal cord or peripheral nerve. The implants, which can be printed, can be simply and rapidly produced, reduce foreign body responses, and/or support linear, aligned host axonal regeneration across a lesion site. Moreover, neural stem cells can be loaded into the implants. The stem cells can support regenerating host axons as they cross the lesion site and bridge beyond, and facilitate functional regeneration in vivo. Thus, disclosed herein are implants fabricated from a mix of degradable materials that reduce the reactive cell layer due to reduction in a foreign body response, allowing host axons to better penetrate and even traverse beyond the lesion

The concentrations of the implant material and the crosslinking density of printed implants can be designed to mimic the mechanical properties of the native neural tissue, e.g., spinal cord tissue, since a mismatch of mechanical properties between an implant and host could lead to compression or laceration at spinal cord interfaces, causing a failure in integration. In some embodiments, the tissue repair scaffold comprises an elastic modulus from about 200 kPa to about 300 kPa as to mimic the mechanical properties of the native neural tissue.

### Device Incorporating Alignment Markers, Microchannel Termination Markers, and Improved Sheath Thickness

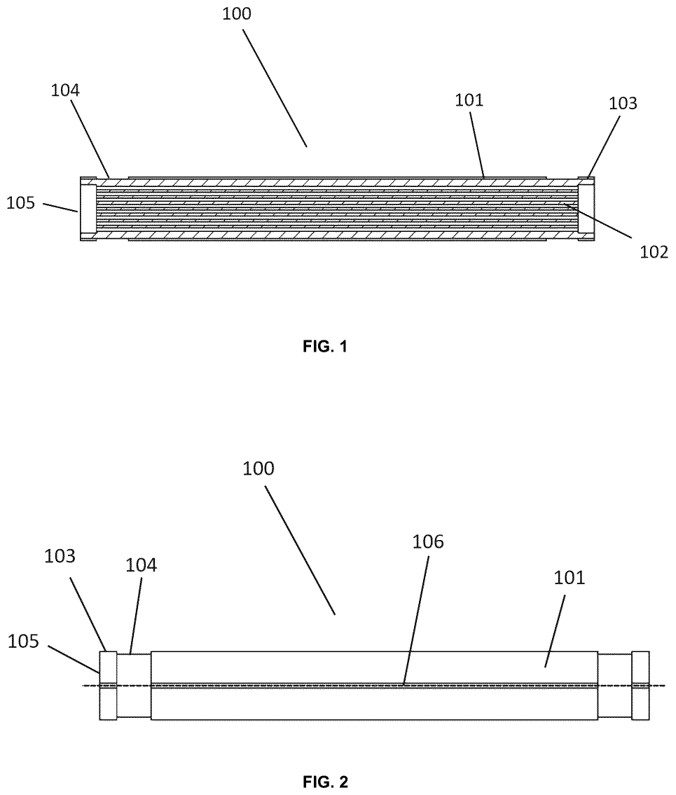

A cross-sectional view of an exemplary, non-limiting embodiment of a tissue repair scaffold device incorporating alignment markers, microchannel termination demarcations, and a sheath thickness of about 500 microns is shown in FIG. 1. FIG. 1 shows a cross section of a tissue repair scaffold 100 which has a sheath 101. The sheath 101 is made of polycaprolactone (PCL) and has a thickness of 500 microns. The sheath 101 also has a porosity of approximately 70% vol. Within the interior of the sheath 101 are a plurality of microchannels 102. The microchannels have a cross sectional diameter of about 200 microns and are also made from PCL. The tissue repair scaffold 100 also comprises an overhang 103 which is useful for suturing the device to a nerve stump. The overhang 103 is a portion of the sheath 101 which extends beyond the terminal point of microchannels 102. This overhang 103 defines a scaffold opening 105 through which a nerve can enter the microchannels 102 and has a length of 1 mm. On the exterior of the sheath 101 is an indented groove are which acts as a microchannel end marker 104. The microchannel end marker 104 has a length of 2 mm. The microchannel end marker 104 allows a surgeon who is implanting the device to readily ascertain where the overhang 103 begins and the microchannels 102 end, thus allowing the surgeon to suture the overhang to the nerve without piercing the microchannels with the suture.

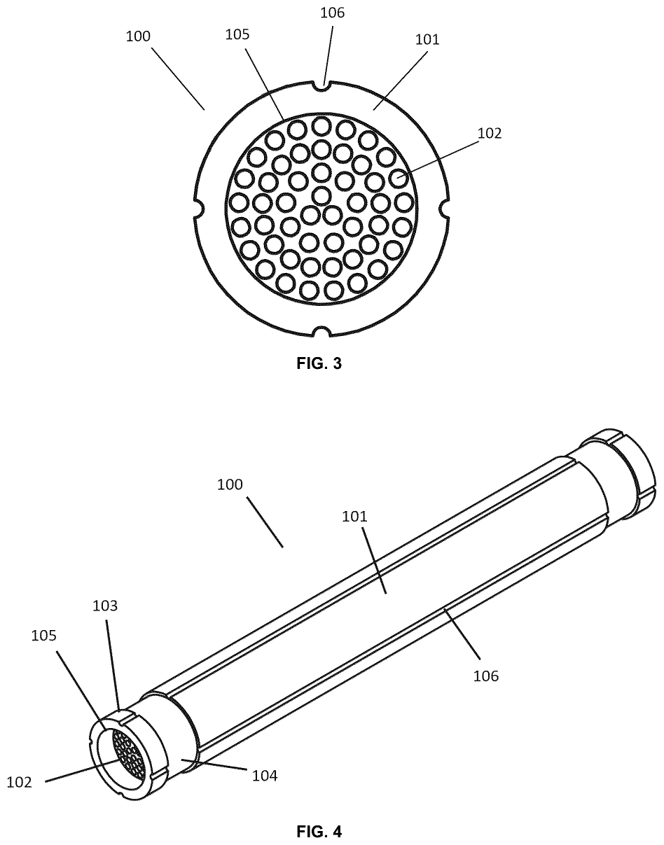

FIG. 2 shows an exterior side view of the scaffold 100. The microchannel end marker 104 can be clearly seen as an indentation or groove structure in the sheath 101 and clearly demarcates where the overhang 103 begins. The scaffold opening 105 is also shown. Also shown on the exterior of sheath 101 is alignment groove 106, which runs substantially from one scaffold opening 105 to the other end of the scaffold 100, except for the portion of sheath 101 encompassed by the end marker 104. As is shown by the dashed line running through FIG. 2, a surgeon implanting the scaffold 100 can tell that the device is properly aligned because alignment groove 106, acting as an alignment marker, is in a straight line.

FIG. 3 shows a front facing view of the tissue repair scaffold 100 looking down the scaffold opening 105. Clearly shown is the sheath 101 with a thickness of 500 microns, as well as the plurality of microchannels 102 disposed within the sheath 101. The microchannels 102 are all substantially parallel with each other. Alignment grooves 106 are shown as indentations into sheath 101. The alignment grooves 106 are substantially parallel with the microchannels 102. The scaffold 100 has four alignment grooves 106 disposed equally dispersed about its circumference.

FIG. 4 shows an angled view of the tissue repair scaffold 100 showing all of the features. The sheath exterior 101 is shown with two of the four alignment grooves 106. The scaffold opening 105 marking the end overhang 103 is shown, as well as how overhang 103 extends beyond the ends of microchannels 102. The edge of microchannel end marker 104 is also shown demarcating the end of microchannels 102 within the interior of the sheath 101.

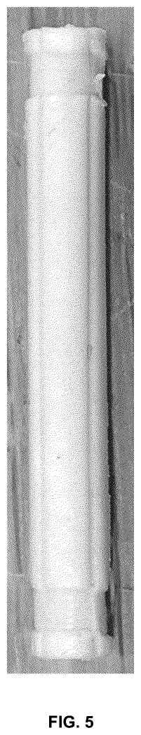

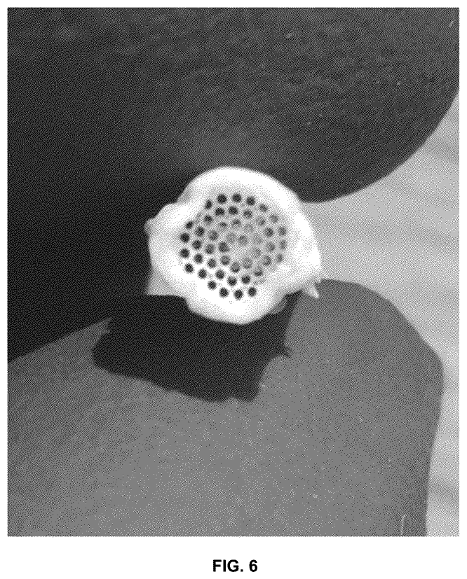

FIG. 5 shows a picture of a tissue repair scaffold as described in FIGS. 1-4 from a side view. Clearly visible are the microchannel end markers and the alignment grooves of the device. The device pictured is about 3 cm in length with an interior sheath diameter of about 3 mm. FIG. 6 shows a picture of the same tissue repair scaffold as FIG. 5 being held in the hand of an individual. The microchannels, alignment grooves, and sheath thickness are all clearly visible.

While device shown in FIGS. 1-6 contains three separate features provided herein (alignment markers, microchannel termination markers, and increased sheath thickness), it is also contemplated that a device could incorporate any one of these features individually or in any combination of two of these features. In some embodiments, a tissue repair scaffold provided herein comprises alignment markers and microchannel termination markers. In some embodiments, a tissue repair scaffold provided herein comprises alignment markers and increased sheath thickness. In some embodiments, a tissue repair scaffold comprises microchannel termination markers and increased sheath thickness. In some embodiments, a tissue repair scaffold comprises alignment markers alone. In some embodiments, a tissue repair scaffold comprises microchannel termination markers alone. In some embodiments, a tissue repair scaffold comprises increased sheath thickness alone.

The present technology thus enables a major advance over existing technologies in surgical repair of injured peripheral nerves. These existing devices consist of only a single open channel (not divided into individual microchannels) in which axons frequently diverge from linear paths, reducing the number of axons that reach the distal end of the scaffold and contribute to nerve repair. Simpler designs like those commercially available more commonly result in painful neuromas and lack of functional improvement because of axon misguidance.

Additionally, the properties of materials out of which existing scaffolds have been fabricated do not adequately support cell and axon attachment. Based on empirical observation after implanting and testing hydrogel nerve regeneration scaffolds, hydrogel-based materials do not exhibit adequate strength to enable the fabrication of thin (<50 μm) wall scaffolds. Yet based on calculations, it appears that wall thicknesses of less than 50 microns are necessary to achieve >80% lumen volume scaffolds that adequately support and promote neural tissue growth. Thus, currently available hydrogel based materials cannot provide scaffolds having adequate strength with advantageous open lumen volume provided by certain aspects of the present teachings. Conversely, the materials provided herein, for example hydrogels comprising a mixture of poly(ethylene glycol) methacrylate and methacrylated gelatin or hydrogels comprising a mixture of poly(ethylene glycol) methacrylate and methacrylated collagen, are mechanically engineered to be stable in vivo. In some embodiments, the use of 3D printing (e.g., digital light processing or other suitable method) allows for high resolution in the printing of the microchannel walls. This high resolution, in some embodiments in conjunction with the materials provided herein, allows for a scaffold to possess the required strength to remain stable in vivo, In some embodiments, the high resolution 3D printing allows for construction of scaffolds with wall thicknesses as low as 10 microns. In some embodiments, the high resolution 3D printing allows for construction of scaffolds with sheath thickness of about 250 microns to about 700 microns as disclosed above.

The present tissue scaffold devices are superior in providing a multi-lumen design that enhances nerve guidance, thereby increasing the total number of axons that regenerate successfully. As a result, such tissue scaffold devices work over long nerve gaps and in more proximal nerve injuries, thereby addressing a great unmet medical need. Further, the tissue scaffolds according to the present disclosure are made from biocompatible and biodegradable materials, such as poly(ethylene glycol) diacrylate, methacryated gelatin, methacrylated collagen, polycaprolactone, or acrylated polycaprolactone, with optimized porosity and surface roughness, providing superior cell adhesion levels and directional cell growth while exhibiting significantly reduced inflammatory response in vivo after implantation. When tested in vivo, the devices of the present disclosure are biocompatible.

In this manner, the tissue scaffold devices according to certain aspects of the present disclosure enable one or more of the following unique features or advantages: a close-packed array of linear microchannels that emulate native nerve organization, microchannels having significant and customizable lengths; hexagonal microchannels to maximize the number of channels within a sheath; thin-walled microchannels to maximize open volume; high open lumen volumes; the scaffold devices comprise biocompatible materials; an ability to control mechanical properties to optimize for strength to minimize wall thickness and sutureability as an outer sheath tube; an ability to control scaffold and sheath porosity to prevent axon penetration while allowing permeation of oxygen and other nutrients; an ability to modify microchannel surface properties to enable cell attachment; a single one-piece sheath and scaffold construction facilitating ease of implantation enabling secure apposition between nerve stumps and scaffold walls; and finally low material and fabrication cost.

Further areas of applicability will become apparent from the description provided herein. The description and specific examples in this summary are intended for purposes of illustration only and are not intended to limit the scope of the present disclosure.

Methods of Treatment

In one aspect, the tissue repair scaffolds provided herein are useful for the regrowth of tissue in a subject in need. In some embodiments, the tissue repair scaffolds are useful for the regrowth of nerve tissue, such as spinal or peripheral nerves. In some embodiments, the tissue repair scaffolds allow for the regrowth and reconnection of severed nerves following implantation into a subject.

In one aspect, provided herein, is a method of restoring nerve function comprising implanting a tissue repair scaffold provided herein into a nerve injury site in a subject in need thereof.

In some embodiments, implanting the tissue repair scaffold allows restoration of nerve function across the injury site. In some embodiments, the restoration is partial restoration of nerve function. In some embodiments, the restoration is complete restoration of nerve function.

In some embodiments, implanting the tissue repair scaffold comprises suturing a first nerve end to the device such that the first nerve end regrows through the microchannel starting at the first end. In some embodiments, impaling the tissue repair scaffold comprises suturing a second nerve end to the device such that the second nerve end regrows through the microchannel starting at the second end. In some embodiments, one of the first nerve or the second nerve end regrow through the entirety of the tissue repair scaffold. In some embodiments, both the first nerve end and the second nerve regrow through the entirety of the tissue repair scaffold. In some embodiments, the first nerve end and the second nerve end reconnect in the interior of the scaffold.

In some embodiments, the nerve is a spinal or peripheral nerve. In some embodiments, the nerve is a spinal nerve. In some embodiments, the nerve is a peripheral nerve.

In some embodiments, the nerve is fully or partially lesioned. In some embodiments, the nerve is fully lesioned. In some embodiments, the nerve is partially lesioned.

In some embodiments, the nerve injury site comprises a gap between nerve ends. In some embodiments, the gap between nerve is from about 0.5 mm to about 10 cm. In some embodiments, the gap between nerve is about 0.1 cm to about 10 cm. In some embodiments, the gap between nerve is about 0.1 cm to about 0.25 cm, about 0.1 cm to about 0.5 cm, about 0.1 cm to about 0.75 cm, about 0.1 cm to about 1 cm, about 0.1 cm to about 2.5 cm, about 0.1 cm to about 5 cm, about 0.1 cm to about 7.5 cm, about 0.1 cm to about 10 cm, about 0.25 cm to about 0.5 cm, about 0.25 cm to about 0.75 cm, about 0.25 cm to about 1 cm, about 0.25 cm to about 2.5 cm, about 0.25 cm to about 5 cm, about 0.25 cm to about 7.5 cm, about 0.25 cm to about 10 cm, about 0.5 cm to about 0.75 cm, about 0.5 cm to about 1 cm, about 0.5 cm to about 2.5 cm, about 0.5 cm to about 5 cm, about 0.5 cm to about 7.5 cm, about 0.5 cm to about 10 cm, about 0.75 cm to about 1 cm, about 0.75 cm to about 2.5 cm, about 0.75 cm to about 5 cm, about 0.75 cm to about 7.5 cm, about 0.75 cm to about 10 cm, about 1 cm to about 2.5 cm, about 1 cm to about 5 cm, about 1 cm to about 7.5 cm, about 1 cm to about 10 cm, about 2.5 cm to about 5 cm, about 2.5 cm to about 7.5 cm, about 2.5 cm to about 10 cm, about 5 cm to about 7.5 cm, about 5 cm to about 10 cm, or about 7.5 cm to about 10 cm. In some embodiments, the gap between nerve is about 0.1 cm, about 0.25 cm, about 0.5 cm, about 0.75 cm, about 1 cm, about 2.5 cm, about 5 cm, about 7.5 cm, or about 10 cm. In some embodiments, the gap between nerve is at least about 0.1 cm, about 0.25 cm, about 0.5 cm, about 0.75 cm, about 1 cm, about 2.5 cm, about 5 cm, or about 7.5 cm. In some embodiments, the gap between nerve is at most about 0.25 cm, about 0.5 cm, about 0.75 cm, about 1 cm, about 2.5 cm, about 5 cm, about 7.5 cm, or about 10 cm.

The subject may be a human or a non-human animal. In some embodiments, the subject is a human. In some embodiments, the subject is a non-human animal. In some embodiments, the subject is a vertebrate animal. In some embodiments, the subject is a mammal. In some embodiments, the subject is a non-human mammal. In some embodiments, the subject is a dog, cat, rodent, horse, cow, pig, primate, or other animal.

### Definitions

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

The term “about” as used herein, when referring to a numerical value or range, allows for a degree of variability in the value or range, for example, within 10%, or within 5% of a stated value or of a stated limit of a range.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

All percent compositions are given as weight-percentages, unless otherwise stated.

All average molecular weights of polymers are weight-average molecular weights, unless otherwise specified.

“Substantially” as the term is used herein means completely or almost completely

By “channel” it is meant that the structure defines an evident longitudinal axis and has an open lumen or hollow core. Channels having such an evident longitudinal axis include an elongated axial dimension, which is longer than the other dimensions (e.g., diameter or width) of the channel. In some embodiments, the elongated channels are linear.

The term “micro-sized,” “micrometer-sized,” or the prefix “micro” affixed to another term (e.g., microchannel) as used herein is generally understood by those of skill in the art to mean having a characteristics on the order of micrometers, (e.g., less than 1 mm, less than 900 micrometers, less than 800 micrometers, less than 700 micrometers, less than 600 micrometers, or less than 500 micrometers).

As used herein, the terms “micrometer” and “microns,” as well as the abbreviation “lam” all have the same meaning and are used interchangeably.

By “biocompatible,” it is meant that a material or combination of materials can be contacted with cells, tissue in vitro or in vivo, or used with mammals or other organisms and has acceptable toxicological properties for contact and/or beneficial use with such cells, tissue, and/or animals.

As used herein, when referring to the “length” of a tissue repair scaffold as provided herein, the length is measured as the length of the one or more microchannels which are disposed within the sheath. Similarly, when a sheath is referred to as having a first end and a second end, it is intended that the first end and second end correspond with the terminal points of any microchannel(s) that are disposed within the sheath, unless specified otherwise. Any additional material which may be part of or attached the sheath which extends beyond these points is referred to as an “overhang structure” or similar term.

### EXAMPLES

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed in practicing the invention. It is intended that the following claims define the scope of the invention and that methods and structures within the scope of these claims and their equivalents be covered thereby.

### Example 1. Manufacturing of Scaffolds with Improved Features for Implantation

Film Material Preparation

A slurry is produced using poly(ε-caprolactone) (Sigma Aldrich, number average molar mass: 80 kDa; PCL). The PCL is solubilized in chloroform at a concentration of 3 wt %. Porosity is introduced by adding milled NaCl as a porogen. The NaCl crystals are prepared by ball milling (Retsch PM100) the NaCl at 400 rpm for 2 hours (intervals of 5 minutes of milling alternating with 5 minutes of rest). The milling produces NaCl having a particle size of approximately 17-20 μm. The NaCl is added to the solubilized PCL to produce a slurry of polymer and salt at a ratio of 30 vol %/70 vol % polymer/salt. The slurry is mixed in a ball mill for 20 minutes (intervals of 2 minutes of mixing alternating with 2 minutes of rest). Subsequently, the PCL+salt slurry is used for creating films which are formed into microchannels and sheaths.

To produce polymer films, the PCL+salt slurry is cast with an automated tape casting coater (MTI Corporation). Slurry is poured onto copper foil (McMaster) and spread using a blade to produce a film having a final dry thickness of 600 μm. The films are air dried and detached from the copper foil by wetting in ethanol. Prior to embossing, the film (PCL+salt) is pressed using an automated calendar, in sequential stages. A portion of the film material used to make the sheath is pressed to a final thickness of 500 μm and the remainder of the film is pressed to a final thickness of about 70 μm.

Embossing Materials Preparation

Stainless-steel wires having an outer diameter of 280 μm are cleaned in ethanol. Clean wires are dipped into a solution of 12 wt % poly(vinyl alcohol) (Sigma Aldrich, 80% hydrolyzed; PVA) in water that is pre-heated to 60° C. Coated wires are hung to dry and then inspected for drips or inhomogeneities in the coating. If drips or inhomogeneities in the coating are found, the wires are excluded from manufacturing. An acetal embossing mold (DELRIN) is machined to have linear grooves separated by 135 μm. Each groove is made with a 0.013-inch endmill, resulting in features that are approximately 300 μm wide and approximately 150 μm deep. A mold for pressing the final multichannel roll together is constructed from aluminum. The mold comprises two pieces registered to produce a single channel of 3.0-mm diameter when fit together.

Embossed Microchannel Preparation

For embossing, two aluminum blocks and the pressing mold are heated to 40-50° C. prior to use. The embossing mold is used at temperatures between approximately 21° C. to 80° C. Sheets of 2-mm foam (Darice) are cut to the size of the embossing mold. Two squares of pressed film (e.g., produced as described above) are cut to size. A first film having a thickness of about 70 μm is laid on the embossing mold and covered by a piece of foam. The sandwich is placed between the pre-heated aluminum blocks and put in a hydraulic press (Carver). The stack is pressed with a force of less than 0.4 metric tons. The block, with film lightly adhering to the surface, is gently removed from the stack. Pre-coated stainless-steel wires are placed in the grooves on the embossing mold and held in place with adhesive tape. In an exemplary embodiment, 16 wires are used to produce a 1.6-mm diameter device. The second film is placed over the top and covered by a piece of foam. The sandwich is placed between the aluminum blocks on the press. The entire stack is pressed with 1.5 metric tons of force for 20 seconds and then removed from the press.

Sheath Preparation

A rectangular sheet of the film having a thickness of 500 μm is cut at the desired size to create a sheath having a desired length and 3 mm internal diameter. The sheet is then wrapped around a stainless steel mandrel with a diameter of 3 mm. The stainless steel mandrel longer than the desired length of the resulting sheath. The ends of the stainless steel mandrel are then placed into metal blocks configured to hold the ends of the steel mandrel which have been heated to about 70° C. for about 5 minutes. The heat transfer from the blocks to the mandrel is sufficient to create a bond between the two ends of the film material to create a single rounded sheath wrapped around the mandrel.

The mandrel is then removed from the heating blocks and placed into an embossing mold configured to prepare four parallel grooves equally spread out about the circumference of the sheath running from the first end to the second end of the sheath and dual 2 mm circumferential grooves into the device spaced 1 mm from the end of the sheath. The mold assembly is then pressed with 1.5 metric tons of force for about 5 minutes and removed from the press. The resulting sheath is stored with the stainless steel mandrel remaining inside until needed for full device preparation.

Device Manufacture

To form a device, the embossed microchannel film with wire spacers is peeled from the embossing block. Excess film on either side of the spacers is cut away with a razor. The device is rolled around an axis parallel to (or substantially parallel to) the axis of the spacers. The roll is placed into a pressing mold pre-heated to 40-50° C. This mold is placed between the two aluminum blocks and pressed to 1.5-2 metric tons of pressure. Once removed from the press, the mold is opened and the roll is removed. The mandrel is then removed from the sheath. The roll is inserted into the sheath and the assembly is immersed in water to remove the porogen (NaCl) and the PVA coating. The assembly is soaked for 1 hour, changing the water at least once. After removal from the water, the spacers are removed with tweezers, and the device is left to air dry. The resulting device has microchannels disposed within the sheath that terminate 1 mm from the ends of the sheath, leaving 1 mm of overhang of sheath material.

### Example 2. Implantation of a Multichannel Nerve Repair Scaffold with Alignment Lines

When performing a gap nerve repair, it is essential to properly align the proximal and distal nerve stumps. Misalignment can result in axons diverting to inappropriate targets which will undermine regeneration and prevent proper connectivity to peripheral organs (e.g., muscles). A nerve repair scaffold provided herein has an inner architecture composed of microchannels that guide regenerating axons. When the surgeon implants the device in the injury gap and attaches the proximal and distal nerve stumps to the device, it is important that the stumps are properly aligned to maximize the potential for successful repair and prevent misalignment on either side of the device. Accordingly, an alignment feature on the outer sheath of the device was developed to facilitate proper alignment.

A multichannel nerve repair scaffold containing an outer sheath that contains four longitudinal lines that run the length of the outer sheath, spaced equally along the circumference of the outer sheath. These lines, which consist of thin surface etchings of the outer sheath, allow the surgeon to align nerve stumps across the gap. These new outer sheaths were tested by an experienced hand and orthopedic surgeon for insertion.

The devices were tested in vivo in a rabbit 3 cm-long sciatic nerve lesion gap model under microscopic guidance. Ease of handling and nerve stump alignment were examined. The devices were tested after hydration using sterile saline for 5 minutes. The following was observed: 1) It was easy to attach the nerve stump to the outer sheath using Ethilon 9-0 suture. 2) The linear etches on the outer sheath surface were successful in guiding nerve implantation at the proximal and distal aspects of the scaffold site. This allowed the surgeon to attach the nerve stumps to the device and rotate them so they aligned with one another, thus mimicking the pre-injured state.

) We conclude that adding thin linear etches to the outer sheath of the scaffold improves alignment ability without any evident loss of mechanical strength or manipulability by the surgeon.

### Example 3. Implantation of a Multichannel Nerve Repair Scaffold with Optimized Outer Sheath Thickness

Nerve gap bridging devices have an outer sheath that extends beyond the ends of the device for suturing to the proximal and distal nerve ends. Feasibility studies for a 3 cm-long multichannel nerve repair scaffold tested a 3 cm-long, 3 mm-inner-diameter scaffold with a 100 micron-thick in a rabbit model of peripheral nerve injury. A total of 18 rabbits received implants of the scaffold into 3 com-long sciatic nerve lesions. The sheaths were manufactured from polycaprolactone and had a porosity of 70% vol.

In this feasibility study, partial biodegradation of the 100 micron-thick outer sheath was observed in 4-of-18 scaffolds after 90 days in vivo. In two of these four cases, this biodegradation resulted in detachment of the scaffold from the distal nerve segment.

In order to correct this defect, scaffolds with outer sheath thicknesses of 200, 300, and 500 microns were prepared made of the same materials. These scaffolds with increased outer sheath thickness were tested by three individuals experienced in the implantation and manipulation of nerve scaffolds.

The devices were tested under a surgical microscope using Ethilon 9-0 sutures. Ease of manipulability, flexibility, suturing, resistance to tear when penetrated by suture, and resistance to tension were examined. Devices were tested both dry and after hydration using sterile saline for 5 minutes or 15 minutes. The following results were observed:

200 microns sheath—The outer sheath at this thickness was readily manipulated and was sufficiently flexible and durable for routine handling. However, when tested with tension applied to a suture placed through the outer sheath, the sheath tore, thus indicating a potentially suboptimal mechanical strength.

300 microns sheath—The outer sheath at this thickness was readily manipulated and was sufficiently flexible and durable for routine handling. When tested with traction applied to a suture placed through the outer sheath, the sheath did not tear; instead, the suture tore, indicating that the mechanical strength of the outer sheath was satisfactory.

500 microns sheath—The outer sheath at this thickness was readily manipulated and was sufficiently flexible and durable for routine handling. When tested with tension applied to a suture placed through the outer heath, the suture tore rather than the outer sheath. This thickness was judged to be easy to handle, sufficiently durable, and resistant to tearing.

The experiment was replicated with hydration for 5 and 15 minutes, with similar results observed each time.

### Example 4. Implantation of a Multichannel Nerve Repair Scaffold with Microchannel End Marker

Nerve gap bridging devices have an outer sheath that extends beyond the ends of the device for suturing to the proximal and distal nerve ends. These outer sheath extensions extend beyond the microchannels disposed on the interior of the device. As the exterior material of the sheath is opaque, the exact location of the microchannels relative to the overhang portion is difficult for an installing surgeon to ascertain. Thus, while attempting to suture the overhang portion to a nerve stump, the surgeon must exercise great care to avoid puncturing the microchannels disposed within the interior of the sheath. If the suture is implanted at the wrong location and punctures one or more of the interior microchannels, the implant will be compromised and potentially ineffective in facilitating nerve regrowth and reconnection of the two nerve ends.

In order to correct this challenge, nerve repair scaffolds are prepared having an outer sheath that extends beyond the ends of the microchannels. The outer sheath has a marker on the exterior that indicates on the exterior of the device where the obscured microchannels on the interior of the device end. The marker is an etching in the sheath which denotes the terminal point of the microchannels on the interior of the device. The etching spans from the point denoting the terminal end of the interior microchannels inward towards the center of the device by a length of 2 mm, which can be easily seen by the installing surgeon.

The scaffolds having the overhang marker are tested under a surgical microscope using Ethilon 9-0 sutures. The devices are tested using an in vivo rabbit 3 cm-long sciatic nerve lesion gap model. The devices are tested after hydration using sterile saline for 5 minutes.

The presence of the outer sheath marker allows the surgeon to readily ascertain where on the device to initiate and complete suturing of the device to the nerve stump without damaging the interior microchannels. The installing surgeon does not have to frequently adjust the device to ascertain where on the interior the channels end. The surgeon is able to install the device into the nerve gap without puncturing any of the interior channels. The presence of the markers does not impact the stability or function of the device.

### Example 5. Multichannel Scaffold for Nerve Injury Repair in Rat Model

In some embodiments, devices are 3D printed from porous PCL, polyethylene glycol, polyethylene glycol diacrylate, gelatin, methacrylated gelatin, collagen, methacrylated collagen, polylactic acid, poly glycolic acid, poly(lactic-co-glycolic acid), polycaprolactone, or acrylated polycaprolactone, or combinations thereof and comprise an improved sheath thickness and a plurality of linear microchannels. The entire device has a length of 10 mm, and has a 1-mm overhang of the outer sheath on either side (e.g., for suturing in place in a subject). The sheath has a wall thickness of 500 μm and a 3 mm internal diameter. The device has an outer sheath that extends beyond the ends of the device for suturing to the proximal and distal nerve ends. These outer sheath extensions extend beyond the microchannels disposed on the interior of the device. The device comprises an elastic modulus from about 200 kPa to about 300 kPa.

As the exterior material of the sheath is opaque, the exact location of the microchannels relative to the overhang portion is difficult for an installing surgeon to ascertain. Thus, while attempting to suture the overhang portion to a nerve stump, the surgeon must exercise great care to avoid puncturing the microchannels disposed within the interior of the sheath. If the suture is implanted at the wrong location and punctures one or more of the interior microchannels, the implant will be compromised and potentially ineffective in facilitating nerve regrowth and reconnection of the two nerve ends.

In order to correct this challenge, nerve repair scaffolds are prepared having an outer sheath that extends beyond the ends of the microchannels. The outer sheath has a first plurality of markers on the exterior that indicates on the exterior of the device where the obscured microchannels on the interior of the device end. The sheath of the device further has a second plurality of markers on the exterior that indicates on the exterior of the device the alignment of the linearly microchannels and the torsion of the device. The markers are an etching in the sheath which denotes the terminal point of the microchannels on the interior of the device, and the alignment of the microchannels therein. The etching spans from the point denoting the terminal end of the interior microchannels inward towards the center of the device by a length of 2 mm, which can be easily seen by the installing surgeon.

The presence of the outer sheath marker allows the surgeon to readily ascertain where on the device to initiate and complete suturing of the device to the nerve stump without damaging the interior microchannels. The installing surgeon does not have to frequently adjust the device to ascertain where on the interior the channels end. The surgeon is able to install the device into the nerve gap without puncturing any of the interior channels. The presence of the markers does not impact the stability or function of the device.

To assess the efficacy of these devices for nerve repair, devices are tested in the rat sciatic nerve model. Animals are housed (e.g., 2-3 per cage) with free access to food and water in facilities approved by the American Association for the Accreditation of Laboratory Animal Care. All animal studies are carried out according to NIH guidelines for laboratory animal care and safety, adhering to protocols approved by the Institutional Animal Care and Use Committee of the VA Healthcare System, San Diego.

To implant devices (n=6), animals are deeply anesthetized (e.g., using ketamine (25 mg/mL), xylazine (1300 mg/mL), and acepromazine (0.25 mg/mL)) before making a 20-mm long incision on the right lateral thigh. The right sciatic nerve trunk is exposed via a lateral gluteal muscle dissection. Epineural connective tissue around the nerve trunk is separated with microscissors and a 6.0-mm long nerve segment is excised. After tissue retraction, the severed nerve stumps are further separated to about 15 mm; they are protected and hydrated with physiological saline. Devices are positioned and attached to the nerve, at either end, using 9-0 Ethicon suture. Devices are positioned to avoid tension at the interfaces of the device and nerve site. Following implantation, muscles are sutured using 5-0 suture and the skin is closed using clips. Antibiotics and analgesics (e.g., banamine (1 mg/kg) and ampicillin (0.2 mg/kg) in Ringer's lactate) are administered for the first 3 days to facilitate recovery from surgery. After 4 weeks, devices are harvested. Animals are perfused with 4% paraformaldehyde (PFA) and the tissue is removed and post-fixed in PFA for another 24 hours followed by 48 hours in 30% sucrose.

After 4 weeks, observations are expected to indicate no sign of degradation of the device. To assess the regeneration of nerves across the lesion site, immunolabeling is performed on histological sections. The tissue is processed for: 1) axon labeling, e.g., to assess axon regeneration through and beyond the injury site (NF200); and 2) Schwann cells (S100).

The microchannels of the device produce an aligned growth of neurites through the length of the scaffold, with nerves exiting the distal side of the implant. Unlike more traditional manufacturing methods (e.g., dip-coating), the high open lumen volume of the devices provided herein allows a greater number of neurons to regenerate due to the reduction of volume taken up by the pore walls. Accordingly, the technology provides for the faster healing of nerve lesions with better functional recovery.

### Example 6. Multichannel Scaffold for Nerve Injury Repair in Rat Model

Multichannel scaffolds prepared by 3D printing method from porous PCL, polyethylene glycol, polyethylene glycol diacrylate, gelatin, methacrylated gelatin, collagen, methacrylated collagen, polylactic acid, poly glycolic acid, poly(lactic-co-glycolic acid), polycaprolactone, or acrylated polycaprolactone, or combinations thereof and were implanted in a 1 cm-long defect in rat sciatic nerve and compared to sural nerve autograft or open tube implant. The scaffolds used in this example approximately 75 microchannels which were each approximately 200 microns in diameter. The scaffolds were 1 cm in length with an outer diameter of 1.7 mm. The device comprises an elastic modulus from about 200 kPa to about 300 kPa.

As the exterior material of the sheath is opaque, the exact location of the microchannels relative to the overhang portion is difficult for an installing surgeon to ascertain. Thus, while attempting to suture the overhang portion to a nerve stump, the surgeon must exercise great care to avoid puncturing the microchannels disposed within the interior of the sheath. If the suture is implanted at the wrong location and punctures one or more of the interior microchannels, the implant will be compromised and potentially ineffective in facilitating nerve regrowth and reconnection of the two nerve ends. Further, the torsion of the device as implanted is also difficult for the installing surgeon to ascertain, as the device is cylindrical in shape and does not obviously indicate if it placed under a torsion.

In order to correct this challenge, nerve repair scaffolds are prepared having an outer sheath that extends beyond the ends of the microchannels. The outer sheath has a first plurality of markers on the exterior that indicates on the exterior of the device where the obscured microchannels on the interior of the device end. The sheath has a second plurality of markers on the exterior surface that indicates on the exterior of the device the alignment of the linearly microchannels and the torsion of the device. The markers are an etching in the sheath which denotes the terminal point of the microchannels on the interior of the device, and the alignment of the microchannels therein. The etching spans from the point denoting the terminal end of the interior microchannels inward towards the center of the device by a length of 2 mm, which can be easily seen by the installing surgeon. The second plurality of markers comprise 4 linear markers which run in a straight line from a proximal end of the implant to a distal end of the implant. The first plurality of markers comprises a circumferential ring around a proximal end of the implant and a distal end of the implant which correspond to the location where the microchannels end and the outer sheath continues.

The presence of the first outer sheath marker allows the surgeon to readily ascertain where on the device to initiate and complete suturing of the device to the nerve stump without damaging the interior microchannels. The presence of the first outer sheath marker allows the surgeon to readily ascertain the alignment of the microchannels contained therein, and any torsion placed upon the device, and easily permits the surgeon to remove any torsion from the device when installing the implant. The installing surgeon does not have to frequently adjust the device to ascertain where on the interior the channels end, or adjust the device to account for torsion placed upon the device. The surgeon is able to install the device into the nerve gap without puncturing any of the interior channels, while maintaining proper alignment (e.g., no torsion) of the linear microchannels therein. The presence of the markers does not impact the stability or function of the device.

Four weeks post-implant multichannel scaffolds support linear alignment and accelerated regeneration of axons across the injury site. Six months post implant multichannel scaffolds showed improved connectivity between spinal cord and gastrocnemius muscle, compared to open tube treatment and comparable to the autograft. In addition, multichannel scaffolds support increased muscle mass, double the amount of increased muscle mass compared to lesion-only or open tube treatments, and comparable to autograft. The multichannel scaffold described herein facilitate superior axonal alignment and faster rate of regeneration across a 1 cm sciatic nerve gap in the rat (shown 4 weeks post injury) compared to an open tube scaffold at the same time point. Further, the implant utilized in this example shows improved connectivity between spinal cord motor neurons and muscle, assessed by injection of retrograde tracer (Cholera Toxin B) into gastrocnemius muscle six months after nerve repair, and result in improved muscle mass. Statistically, the multichannel scaffold is as effective as a sural nerve autograft. N=11 animals per group.

The biomimetic scaffold with microchannels disclosed herein has improved anatomical and electrophysiological connectivity across a sciatic nerve injury site and supports recovery of motor function.

1.-93. (canceled)

94. A tissue repair scaffold comprising:
a sheath having a first end and a second end;
a microchannel disposed within an interior of the sheath, wherein the microchannel traverses the sheath from the first end to the second end; and
a marker disposed on an exterior surface of the sheath running substantially from the first end to the second end.

95. The tissue repair scaffold of claim 94, wherein the marker comprises a groove.

96. The tissue repair scaffold of claim 94, wherein the marker comprises a feature disposed on an exterior surface of the sheath running substantially from the first end to the second end.

97. The tissue repair scaffold of claim 94, wherein the exterior surface of the sheath comprises at least 2, 3, or 4 grooves disposed thereon.

98. The tissue repair scaffold of claim 97, wherein the grooves are about evenly dispersed about a circumference of the sheath.

99. The tissue repair scaffold of claim 94, further comprising a plurality of markers, wherein the markers indicate the alignment of the scaffold and/or torsion placed upon the scaffold.

100. The tissue repair scaffold of claim 95, wherein the groove has a width or depth of at least about 50 microns.

101. The tissue repair scaffold of claim 94, wherein the marker is substantially parallel with the microchannel.

102. The tissue repair scaffold of claim 94, wherein the tissue repair scaffold comprises a plurality of microchannels disposed within the interior of the sheath.

103. The tissue repair scaffold of claim 102, wherein the plurality of microchannels are substantially parallel.

104. The tissue repair scaffold of claim 102, wherein the marker is substantially parallel to each of the plurality of microchannels.

105. The tissue repair scaffold of claim 94, wherein a length of the scaffold is about 0.5 cm to about 30 cm.

106. The tissue repair scaffold of claim 94, wherein the inner diameter of the sheath is about 1.5 mm to about 10 mm.

107. The tissue repair scaffold of claim 94, wherein the sheath, the microchannel, or both are made from a biodegradable polymer.

108. The tissue repair scaffold of claim 107, wherein the biodegradable polymer is selected from polyethylene glycol, polyethylene glycol diacrylate, gelatin, methacrylated gelatin, collagen, methacrylated collagen, polylactic acid, poly glycolic acid, poly(lactic-co-glycolic acid), polycaprolactone, and acrylated polycaprolactone, or any combination thereof.

109. The tissue repair scaffold of claim 107, wherein the biodegradable polymer is polycaprolactone.

110. The tissue repair scaffold of claim 94, wherein walls of the microchannel comprise hydrogel materials.

111. The tissue repair scaffold of claim 94, further comprising an overhang structure, wherein the overhang structure extends beyond a first end or the second of a microchannel.

112. The tissue repair scaffold of claim 94, wherein the sheath has a thickness of at least about 250 microns.

113. A method of restoring nerve function in a subject comprising:
a. implanting a tissue repair scaffold into the subject, the tissue repair scaffold comprising:
i. a sheath having a first end and a second end;
ii. a microchannel disposed within an interior of the sheath, wherein the microchannel traverses the sheath from the first end to the second end;
iii. a marker disposed on an exterior surface of the sheath running substantially from the first end to the second end;
iv. an overhang structure, wherein the overhang structure extends beyond the first end or the second of the microchannel;
b. aligning the marker with a neural tissue of a subject and aligning the marker from the first end to the second end of the sheath such that there is no torsion on the device and microchannel disposed within the interior of the sheath; and
c. attaching the overhang to the subject proximal to the neural tissue, thereby securing the device to the subject proximal to the neural tissue such that there is no torsion on the device and microchannel disposed within the interior of the sheath is aligned with the neural tissue.
